(19) United States
(12) Patent Application Publication
(10) Pub. No.: US 2014/0214129 A1
(43) Pub. Date: Jul. 31, 2014

(54) ENERGY EFFICIENT NEUROMODULATION

(71) Applicants: Jonathan J. Waataja, Plymouth, MN (US); Adrianus P. Donders, Andover, MN (US)

(72) Inventors: Jonathan J. Waataja, Plymouth, MN (US); Adrianus P. Donders, Andover, MN (US)

(73) Assignee: ENTEROMEDICS INC., St. Paul, MN (US)

(21) Appl. No.: 14/163,503

(22) Filed: Jan. 24, 2014

Related U.S. Application Data
(60) Provisional application No. 61/757,575, filed on Jan. 28, 2013.

(51) Int. Cl. A61N 1/36; A61N 1/372

(52) U.S. Cl. CPC A61N 1/36007; A61N 1/37247
USPC 607/59; 607/60; 607/116

(57) ABSTRACT

A therapy system for applying an electrical signal to a target nerve includes an electrode, an implantable component and an external component. The electrode has an impedance of at least about 2000 ohms. The electrical signal is applied using constant current or constant voltage.

### CROSS REFERENCE

This application claims priority to U.S. application No. 61/757,575, filed Jan. 28, 2013, which application is hereby incorporated by reference.

### BACKGROUND OF THE INVENTION

Obesity, diabetes, hypertension, and other gastrointestinal disorders are serious health conditions that lead to increased morbidity and mortality. For example, over the last decade, the prevalence of obesity has increased more than 80%, representing an estimated 43 million adults in 2002. (Mokdad A H, et al, The spread of the obesity epidemic in the United States, 1991-1998. JAMA 1999; (282):1519-22) In terms of mortality, an estimated 280,000 to 325,000 adults in the United States die each year from causes related to obesity (Allison D B et al, Annual deaths attributable to obesity in the United States. JAMA 1999; 282:1530-8) More importantly, excess weight has been positively correlated with years of life lost. (Fontaine K R et al., Years of life lost due to obesity. JAMA 2003; (289):187-93). Several other diseases have comorbidity with obesity such as metabolic syndrome, type II diabetes, heart disease, and hypertension.

Thus, there remains a need to develop effective treatments for conditions such as diabetes, hypertension, obesity, heart disease, and metabolic syndrome.

### SUMMARY OF THE INVENTION

According to one aspect of the disclosure, a therapy system is disclosed for applying therapy to an internal anatomical feature of a patient. The system includes at least one high impedance electrode for implantation within the patient and placement at the anatomical feature (e.g., a nerve) for applying the therapy signal to the feature upon application of a treatment signal to the electrode. An implantable component is placed in the patient's body beneath a skin layer and coupled to the electrode for delivery of an electrical signal using a selected current or a selected voltage. The signal may be monophasic or biphasic. The implantable component includes an implanted antenna. An external component has an external antenna for placement above the skin and adapted to be electrically coupled to the implanted antenna.

In embodiments, a system for applying therapy to a target nerve of a subject comprises at least two electrodes, each having an impedance of at least 2000 ohms configured to be implanted within a body of the subject and placed at the target nerve, an implantable component for placement in the body of the subject, the implantable component being configured to generate an electrical signal at a selected voltage or a selected current, wherein the electrical signal is selected to modulate activity on the target nerve, the implantable component being coupled to an implanted antenna; an external component including an external antenna configured to be placed above the skin layer and adapted to communicate with the implanted antenna. In embodiments, the system of claim 1, further comprises an external programmer configured to communicatively couple to the external component, the external programmer being configured to provide therapy instructions to the external component, wherein the external component is configured to send the therapy instructions to the implantable component via the external antenna and the implanted antenna.

Another aspect of the disclosure provides a method of treating a disorder in a subject comprising applying an electrode to a target nerve, wherein the electrode has an impedance of at least 2000 ohms and is operatively coupled to an implantable neuroregulator; applying a therapy cycle to the target nerve, wherein the therapy cycle comprises applying an electrical signal at a selected current or selected voltage to the electrode intermittently, and is selected to downregulate or upregulate activity on the target nerve.

In embodiments, a method of treating a disorder in a subject comprises applying at least two electrodes to a target nerve, wherein each electrode has an impedance of at least 2000 ohms and is operatively coupled to an implantable neuroregulator; and applying a therapy cycle to the target nerve, wherein the therapy cycle comprises applying an electrical signal at a selected voltage or selected current to the electrode intermittently, wherein the electrical signal is selected to modulate activity on the target nerve. In embodiments, the disorder is selected from the group consisting of obesity, metabolic syndrome, diabetes, hypertension, inflammatory bowel disease, pancreatitis, and bulimia.

### III. BRIEF DESCRIPTION OF THE DRAWINGS

FIG. 1 is a schematic representation of a therapy system having features that are examples of inventive aspects of the principles of the present invention, the therapy system including a neuroregulator and an external charger;

FIG. 2A is a plan view of an implantable neuroregulator for use in the therapy system of FIG. 1 according to aspects of the present disclosure;

FIG. 2B is a plan view of another implantable neuroregulator for use in the therapy system of FIG. 1 according to aspects of the present disclosure.

FIG. 3A is a block diagram of a representative circuit module for the neuroregulator of FIG. 2A and FIG. 2B according to aspects of the present disclosure;

FIG. 3B is a block diagram for a low power arbitrary waveform generator intended for implantable therapeutic devices. Some of the functionality is optional such as the memory and telemetry blocks;

FIG. 4 is a block diagram of a circuit module for an external charger for use in the therapy system of FIG. 1 according to aspects of the present disclosure;

FIG. 5 shows electrode configuration and the HFAC waveform. (A) Schematic representation of relative positions of stimulating (S), HFAC, and recording (R) electrodes on the isolated vagus nerve. (B). The HFAC waveform has charge-balanced alternating current pulses delivered at 5000 Hz for 1 minute. Pulse width (w, 90 or 10 μs) was constant, and an off-time of 10 or 90 μs was included in each cycle. The current amplitude (a) was varied randomly. (C) Schematic representation of a simplified electrode system. The electrical representation of the electrode to nerve interface is shown. The electrode to nerve capacitance is generally high (in the order of tens to hundreds of pF), while the resistance is low (in the order of tens of Ohms);

FIG. 6 shows A) Plots of current versus time (i) and voltage versus time (ii) for a constant current device. Note that voltage quickly increases in (ii) due to current charging the capacitance of the nerve and electrode system. Then the voltage continues to rise slowly due to current charging the electrode to nerve capacitance;

B) Plot of voltage versus time (i) and current versus time (ii and iii) for a constant voltage device with low (i) and high (iii) impedance electrodes. Note that following the initial current spike in (ii) charging the capacitance of the nerve and electrode system, the remaining current will be essentially determined by the parallel resistance of the nerve. In (iii) the current goes down to a lower level due to the additional resistance of the electrode to nerve interface.

C) Vectors representing the resistance (R), capacitance (C) and impedance (Z) of uncoated (i) versus coated (ii) electrodes. Note the large increase in resistance with the coated electrodes.

FIG. 7 shows that decreasing the C-wave Amplitude was dependent on current and impedance. Plot of C-wave amplitude following conduction block versus current with three different impedance ranges. The dashed lines indicate the effective current to attenuate 50% of the evoked C-wave for each impedance range. Note that with higher impedances, less current was required to attenuate the C-wave.

FIG. 8 shows that attenuation of evoked C-waves was dependent on the voltage across the HFAC electrodes. Plot of the C-wave amplitude following block versus voltage. The dashed lines indicate the voltage required to attenuate 50% of the C-wave.

FIG. 9 shows a schematic of the circuit used to create a constant voltage waveform from a constant current source. Using Ohms law the amount of voltage across the electrodes could be calculated as a function of the current amplitude, taking the electrode impedance into consideration.

FIG. 10 shows a plot of impedances across the HFAC electrodes versus current flowing across the HFAC electrodes which induced a 50% block. Note: as the impedances increased less current was required to induce conduction block.

FIG. 11 shows A) Plot of voltage versus time across the series resistor with a 90 μS pulse width. The first peak was a result of the device shorting out to assure there was no DC offset. The second spike was due to current charging the capacitance of a high impedance electrode. Note that following the second spike the voltage dropped to nearly zero indicating very little current flowing through the nerve.

B) Plot taken with a current probe, which demonstrated nearly zero current flowing through the nerve. The first peak was a result of the device shorting out to assure there was no DC offset. The second spike was due to current charging the capacitance of the high impedance electrode. Note that following the second spike the current dropped to nearly zero. In this experiment a 90 μS pulse width was applied.

FIG. 12 shows a plot of voltage versus time across the series resistor using a 100 μS pulse width. The first spike was due to current charging the capacitance of the high impedance electrode. The second spike was due to current charging the capacitance of electrode in the opposite direction. Note that following the first spike the voltage dropped to nearly zero indicating very little current flowing through the nerve.

FIG. 13 shows a graph of pulse width versus Aδ- or Aα-wave amplitude following a 5000 Hz pulse.

FIG. 14 shows a high impedance electrode configuration. i) Side view of a high impedance electrode design. The helical portion of the electrode is placed around the nerve. ii) Top front view of the high impedance electrode. iii) Top back view of high impedance electrode. The lighter colored stripes represent coated electrodes.

FIG. 15 shows an embodiment of an electrode.

FIG. 16 shows an embodiment of an electrode including a silicon cuff with two parallel plates along the interior of the cuff for contact with the nerve.

### IV. DETAILED DESCRIPTION

In embodiments, methods and systems involve using high impedance electrodes to establish an electrical field across the nerve. Since the electrode is insulated the amount of field sustaining current is minimized. In this case, current charges the electrode capacitance with very little current flowing through the nerve. The applied voltage differential would surround the nerve driving voltage gated channels on individual cells open or closed. In this case, a voltage is applied to first charge the capacitance of the electrode. Following this, little current is flowing through the nerve because the electrodes are coated with a limited-conductive material which minimizes the amount of field sustaining current resulting in energy savings and increased safety. This is different than traditional methods using a low impedance electrode with the requirement of large currents flowing through the resistance of the nerve to induce a voltage differential. Use of an insulating electrode nerve interface provides an electrical field that can be sustained using a very low charge.

The application of such electrodes has wide applicability to a number of conditions employing electric signals to modulate nerve activity. For example, systems having a voltage or current regulated source with high impedance electrodes are useful in the application of an electrical signal to at least partially downregulate activity on a target nerve such as the vagus nerve, renal nerve, celiac nerve, cranial nerves and splanchnic nerve. In other embodiments, the signal may upregulate activity on a target nerve such as the glossopharyngeal nerve and baroreceptors. Modulation of activity on the target nerve can be used to treat a variety of conditions such as obesity, diabetes, hypertension, metabolic conditions, pancreatitis, inflammatory bowel disease, bulimia, dysmotility disorders, and combinations thereof.

In embodiments, it is desirable to provide an implantable device that is able to deliver an electrical signal to a nerve to at least partially modulate the nerve activity while minimizing the power requirements. Minimizing the power requirements decreases the size of the battery allowing for construction of a smaller device, prolongs life of the battery in the device and requires shorter charging times for the battery. Use of an electrode that has high impedance provides for application of an electric signal at a selected voltage or current with very low power requirements and with a low risk of any tissue damage. Such devices also have enhanced compatibility with magnetic resonance imaging. Devices and methods are described herein that provide such an electrical signal.

With reference now to the various drawing figures in which identical elements are numbered identically throughout, a description of embodiments of the present disclosure will now be described.

A. Therapy System

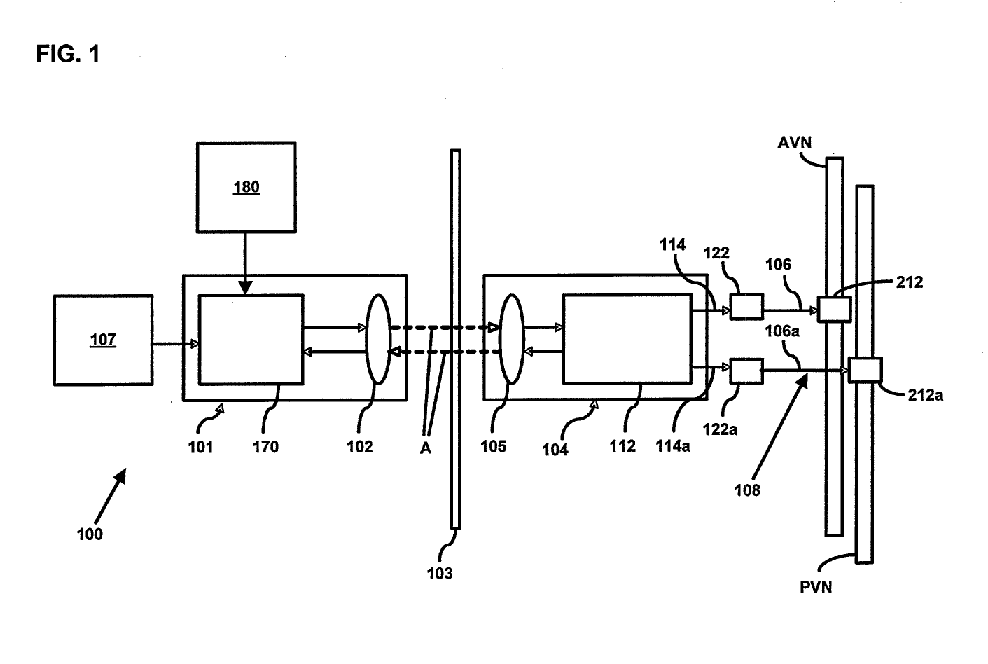

FIG. 1 schematically illustrates a therapy system 100. The therapy system 100 includes a neuroregulator 104, an electrical lead arrangement 108, and an external charger 101. The neuroregulator 104 is adapted for implantation within a patient. As will be more fully described herein, the neuroregulator 104 typically is implanted just beneath a skin layer 103.

The neuroregulator 104 is configured to connect electrically to the lead arrangement 108. In general, the lead arrangement 108 includes two or more electrical lead assemblies 106, 106a. In embodiments, a single lead comprises at least two electrodes. In other embodiments, each lead comprises a single electrode. In the example shown, the lead arrangement 108 includes two identical (bipolar) electrical lead assemblies 106, 106a. The neuroregulator 104 generates therapy signals and transmits the therapy signals to the lead assemblies 106, 106a.

The lead assemblies 106, 106a up-regulate and/or down-regulate nerves of a patient based on the therapy signals provided by the neuroregulator 104. In an embodiment, the lead assemblies 106, 106a include distal electrodes 212, 212a, which are placed on one or more nerves of a patient. For example, the electrodes 212, 212a may be individually placed on the anterior vagal nerve AVN and posterior vagal nerve PVN, respectively, of a patient. For example, the distal electrodes 212, 212a can be placed just below the patient's diaphragm. In other embodiments, however, fewer or more electrodes can be placed on or near fewer or more nerves. In embodiments, the electrodes have an impedance of at least about 2000 Ohms.

The external charger 101 includes circuitry for communicating with the implanted neuroregulator 104. In general, the communication is transmitted across the skin 103 along a two-way signal path as indicated by arrows A. Example communication signals transmitted between the external charger 101 and the neuroregulator 104 include treatment instructions, patient data, and other signals as will be described herein. Energy also can be transmitted from the external charger 101 to the neuroregulator 104 as will be described herein.

In the example shown, the external charger 101 can communicate with the implanted neuroregulator 104 via bidirectional telemetry (e.g. via radiofrequency (RF) signals). The external charger 101 shown in FIG. 1 includes a coil 102, which can send and receive RF signals. A similar coil 105 can be implanted within the patient and coupled to the neuroregulator 104. In an embodiment, the coil 105 is integral with the neuroregulator 104. The coil 105 serves to receive and transmit signals from and to the coil 102 of the external charger 101.

For example, the external charger 101 can encode the information as a bit stream by amplitude modulating or frequency modulating an RF carrier wave. The signals transmitted between the coils 102, 105 preferably have a carrier frequency of about 6.78 MHz. For example, during an information communication phase, the value of a parameter can be transmitted by toggling a rectification level between half-wave rectification and no rectification. In other embodiments, however, higher or lower carrier wave frequencies may be used.

In an embodiment, the neuroregulator 104 communicates with the external charger 101 using load shifting (e.g., modification of the load induced on the external charger 101). This change in the load can be sensed by the inductively coupled external charger 101. In other embodiments, however, the neuroregulator 104 and external charger 101 can communicate using other types of signals.

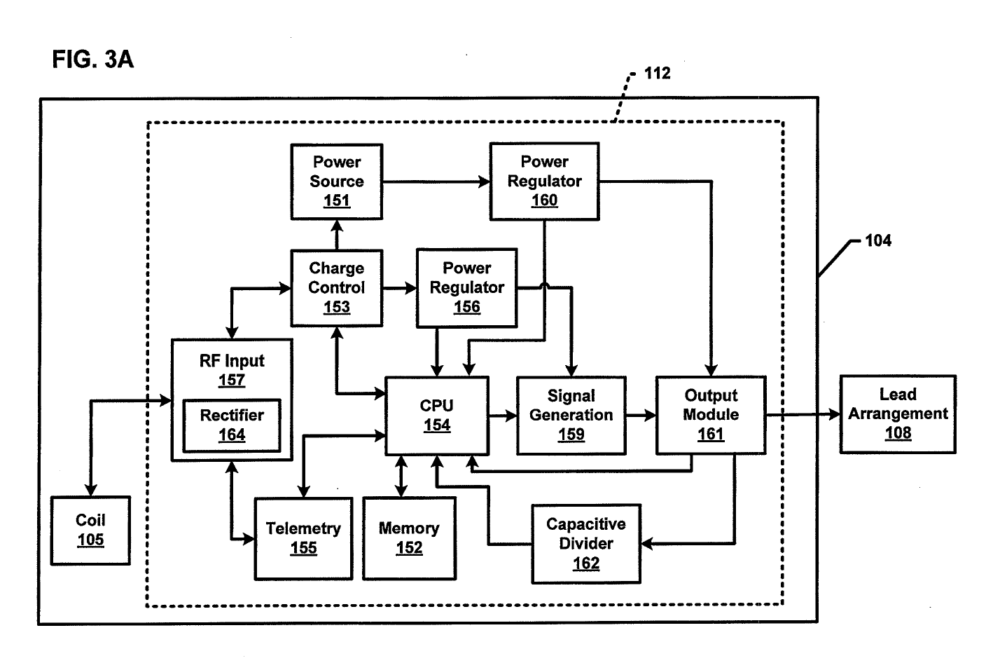

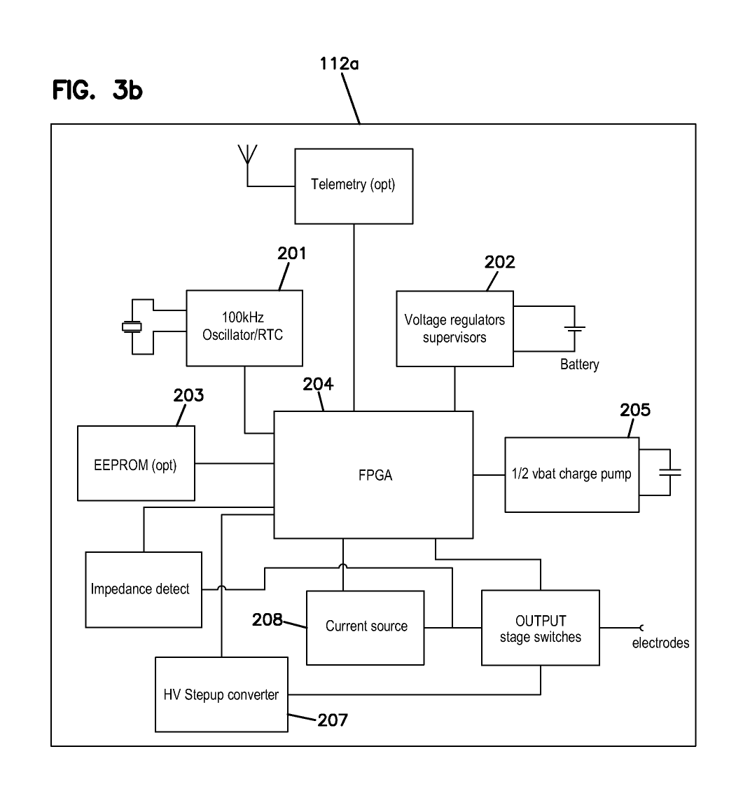

In an embodiment, the neuroregulator 104 receives power to generate the therapy signals from an implantable power source 151 (see FIG. 3A), such as a battery. In a preferred embodiment, the power source 151 is a rechargeable battery. In some embodiments, the power source 151 can provide power to the implanted neuroregulator 104 when the external charger 101 is not connected. In other embodiments, the external charger 101 also can be configured to provide for periodic recharging of the internal power source 151 of the neuroregulator 104. In an alternative embodiment, however, the neuroregulator 104 can entirely depend upon power received from an external source. For example, the external charger 101 can transmit power to the neuroregulator 104 via the RF link (e.g., between coils 102, 105).

In embodiments, the neuroregulator can be powered by a rechargeable battery, which is periodically charged by the application of the mobile charger, the latter being placed in close proximity to the implanted neurorgulator. Alternatively, the neuroregulator can be directly powered by RF energy provided by the mobile charger. The choice of the mode of providing power is made via a setting of the mobile charger, or via the clinician programmer. In a further embodiment, charging of the rechargeable battery in the neuroregulator, can be achieved by application of remote wireless energy. (Grajski et al, IEEE Microwave Workshop series on Innovative Wireless Power Transmission:Technology, Systems, and Applications, 2012 published on a4wp.org).

In some embodiments, the neuroregulator 104 initiates the generation and transmission of therapy signals to the lead assemblies 106, 106a. In an embodiment, the neuroregulator 104 initiates therapy when powered by the internal battery 151. In other embodiments, however, the external charger 101 triggers the neuroregulator 104 to begin generating therapy signals. After receiving initiation signals from the external charger 101, the neuroregulator 104 generates the therapy signals and transmits the therapy signals to the lead assemblies 106, 106a.

In other embodiments, the external charger 101 also can provide the instructions according to which the therapy signals are generated (e.g., pulse-width, amplitude, and other such parameters). In a preferred embodiment, the external charger 101 includes memory in which individual parameters, programs, and/or therapy schedules can be stored for transmission to the neuroregulator 104. Selection of those parameters can be made by a user on a user interface. In embodiments, those parameters include pulse width, constant voltage settings, constant current settings, frequency, and electrode size. For example, one such program can involve selection of a frequency of about 200-5000 Hz, selection of a constant voltage of about 1-20 volts, and selection of a variety of pulse widths ranging from about 10 microseconds to 100 microseconds. The external charger 101 also can enable a user to select a parameter/program/therapy schedule as displayed on a user interface, and then stored in memory for transmission to the neuroregulator 104. In another embodiment, the external charger 101 can provide treatment instructions with each initiation signal.

Typically, each of the parameters/programs/therapy schedules stored on the external charger 101 can be adjusted by a physician to suit the individual needs of the patient. For example, a computing device (e.g., a notebook computer, a personal computer, etc.) 107 can be communicatively connected to the external charger 101. With such a connection established, a physician can use the computing device 107 to program parameters and/or therapies into the external charger 101 for either storage or transmission to the neuroregulator 104.

The neuroregulator 104 also may include memory 152 (see FIG. 3A) in which treatment instructions and/or patient data can be stored. For example, the neuroregulator 104 can store therapy programs or individual parameters indicating what therapy should be delivered to the patient. The neuroregulator 104 also can store patient data indicating how the patient utilized the therapy system 100 and/or reacted to the delivered therapy.

In what follows, the focus of the detailed description is the embodiment in which the neuroregulator 104 contains a rechargeable battery 151 from which the neuroregulator 104 may draw power (FIG. 3A).

### 1. System Hardware Components

a. Neuroregulator

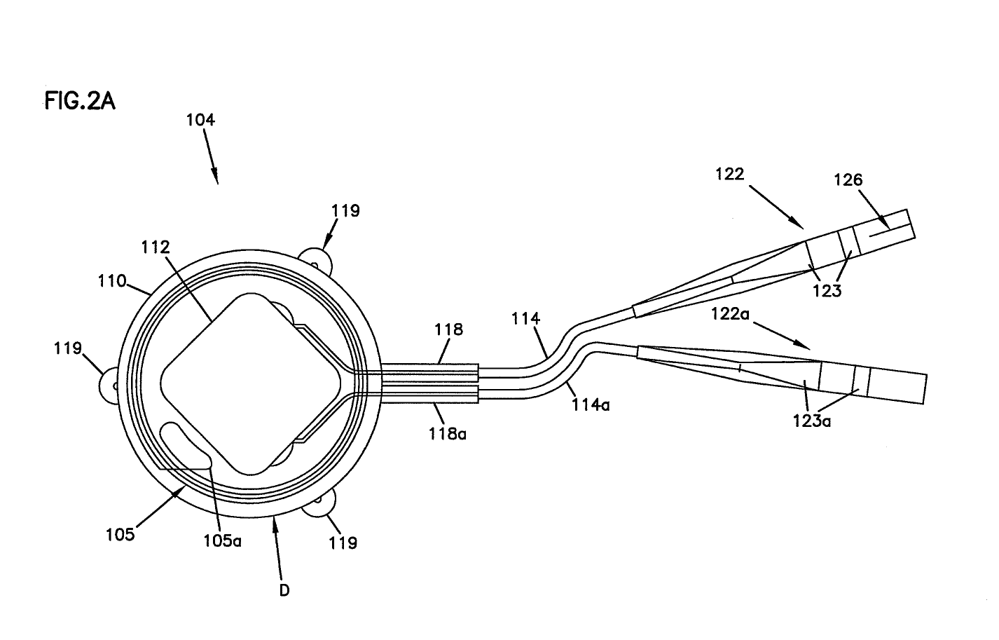

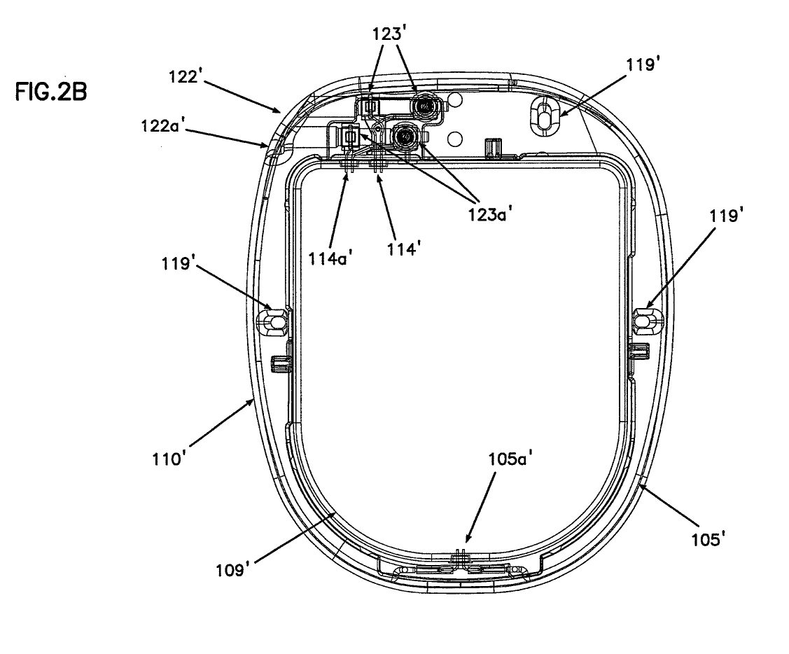

Different embodiments of the neuroregulator 104, 104′ are illustrated schematically in FIGS. 2A and 2B, respectively. The neuroregulator 104, 104′ is configured to be implanted subcutaneously within the body of a patient. In embodiments, the neuroregulator 104, 104′ is implanted subcutaneously on the thoracic sidewall in the area slightly anterior to the axial line and caudal to the arm pit. In other embodiments, alternative implantation locations may be determined by the implanting surgeon.

Typically, the neuroregulator 104, 104′ is implanted parallel to the skin surface to maximize RF coupling efficiency with the external charger 101. In an embodiment, to facilitate optimal information and power transfer between the internal coil 105, 105′ of the neuroregulator 104, 104′ and the external coil 102 of the external charger 101, the patient can ascertain the position of the neuroregulator 104, 104′ (e.g., through palpation or with the help of a fixed marking on the skin). In an embodiment, the external charger 101 can facilitate coil positioning.

As shown in FIGS. 2A and 2B, the neuroregulator 104, 104′ generally includes a housing 109, 109′ overmolded with the internal coil 105, 105′, respectively. The overmold 110, 110′ of the neuroregulator 104, 104′ is formed from a bio-compatible material that is transmissive to RF signals (i.e., or other such communication signals). Some such bio-compatible materials are well known in the art. For example, the overmold 110, 110′ of the neuroregulator 104, 104′ may be formed from silicone rubber or other suitable materials. The overmold 110, 110′ also can include suture tabs or holes 119, 119′ to facilitate placement within the patient's body.

The housing 109, 109′ of the neuroregulator 104, 104′ also may contain a circuit module, such as circuit 112 (see FIGS. 1, 3A, and 3B), to which the coil 105, 105′ may be electrically connected along a path 105a, 105a′. The circuit module within the housing 109 may be electrically connected to a lead assembly, for example, the lead assemblies 106, 106a (FIG. 1) through conductors 114, 114a. In other embodiments, a single lead may be employed. In the example shown in FIG. 2A, the conductors 114, 114a extend out of the housing 109 through strain reliefs 118, 118a. Such conductors 114, 114a are well known in the art.

The conductors 114, 114a terminate at connectors 122, 122a, which are configured to receive or otherwise connect the lead assemblies 106, 106a (FIG. 1) to the conductors 114, 114a. By providing connectors 122, 122a between the neuroregulator 104 and the lead assemblies 106, 106a, the lead assemblies 106, 106a may be implanted separately from the neuroregulator 104. Also, following implantation, the lead assemblies 106, 106a may be left in place while the originally implanted neuroregulator 104 is replaced by a different neuroregulator.

As shown in FIG. 2A, the neuroregulator connectors 122, 122a can be configured to receive connectors 126 of the lead assemblies 106, 106a. For example, the connectors 122, 122a of the neuroregulator 104 may be configured to receive pin connectors (not shown) of the lead assemblies 106, 106a. In another embodiment, the connectors 122, 122a may be configured to secure to the lead assemblies 106, 106a using set-screws 123, 123a, respectively, or other such fasteners. In a preferred embodiment, the connectors 122, 122a are well-known IS-1 connectors. As used herein, the term “IS-1” refers to a connector standard used by the cardiac pacing industry, and is governed by the international standard ISO 5841-3.

In the example shown in FIG. 2B, female connectors 122′, 122a′ configured to receive the leads 106, 106a are molded into a portion of the overmold 110′ of the neuroregulator 104′. The leads connectors 126 are inserted into these molded connectors 122′, 122a′ and secured via setscrews 123′, 123a′, seals (e.g., Bal Seals®), and/or another fastener.

The circuit module 112 (see FIGS. 1, 3A, and 3B) is generally configured to generate therapy signals and to transmit the therapy signals to the lead assemblies 106, 106a. The circuit module 112 also may be configured to receive power and/or data transmissions from the external charger 101 via the internal coil 105. The internal coil 105 may be configured to send the power received from the external charger to the circuit module 112 for use or to the internal power source (e.g., battery) 151 of the neuroregulator 104 to recharge the power source 151.

Block diagrams of example circuit modules 112, 112a are shown in FIGS. 3A, 3B, respectively. Either circuit module 112, 112a can be utilized with any neuroregulator, such as neuroregulators 104, 104′ described above. The circuit modules 112, 112a differ in that the circuit module 112a may be operated directly from a field programmable gate array(204), without the presence of a micro controller reducing its power consumption, and the circuit module 112 does not. Power operation for circuit module 112 may be provided by the external charger 101 or by the internal power source 151. Either circuit module 112, 112a may be used with either neuroregulator 104, 104′ shown in FIGS. 2A, 2B.

The circuit module 112 includes an RF input 157 including a rectifier 164. The rectifier 164 converts the RF power received from the internal coil 105 into DC electric current. Direct current can then be used to provide for a potential on the high impedance electrode. Alternatively, alternating current can be used to provide a selectable but constant voltage or current. Circuitry for constant voltage or constant current devices is known to those of skill in the art.

For example, the RF input 157 may receive the RF power from the internal coil 105, rectify the RF power to a DC power, and transmit the DC current to the internal power source 151 for storage. In one embodiment, the RF input 157 and the coil 105 may be tuned such that the natural frequency maximizes the power transferred from the external charger 101.

In an embodiment, the RF input 157 can first transmit the received power to a charge control module 153. The charge control module 153 receives power from the RF input 157 and delivers the power where needed through a power regulator 156. For example, the RF input 157 may forward the power to the battery 151 for charging or to circuitry for use in creating therapy signals as will be described below. When no power is received from the coil 105, the charge control 153 may draw power from the battery 151 and transmit the power through the power regulator 160 for use. For example, a central processing unit (CPU) 154 of the neuroregulator 104 may manage the charge control module 153 to determine whether power obtained from the coil 105 should be used to recharge the power source 151 or whether the power should be used to produce therapy signals. The CPU 154 also may determine when the power stored in the power source 151 should be used to produce therapy signals.

The transmission of energy and data via RF/inductive coupling is well known in the art. Further details describing recharging a battery via an RF/inductive coupling and controlling the proportion of energy obtained from the battery with energy obtained via inductive coupling can be found in the following references, all of which are hereby incorporated by reference herein: U.S. Pat. No. 3,727,616, issued Apr. 17, 1973, U.S. Pat. No. 4,612,934, issued Sep. 23, 1986, U.S. Pat. No. 4,793,353, issued Dec. 27, 1988, U.S. Pat. No. 5,279,292, issued Jan. 18, 1994, and U.S. Pat. No. 5,733,313, issued Mar. 31, 1998.

In general, the internal coil 105 may be configured to pass data transmissions between the external charger 101 and a telemetry module 155 of the neuroregulator 104. The telemetry module 155 generally converts the modulated signals received from the external charger 101 into data signals understandable to the CPU 154 of the neuroregulator 104. For example, the telemetry module 155 may demodulate an amplitude modulated carrier wave to obtain a data signal. In one embodiment, the signals received from the internal coil 105 are programming instructions from a physician (e.g., provided at the time of implant or on subsequent follow-up visits). The telemetry module 155 also may receive signals (e.g., patient data signals) from the CPU 154 and may send the data signals to the internal coil 105 for transmission to the external charger 101.

The CPU 154 may store operating parameters and data signals received at the neuroregulator 104 in an optional memory 152 of the neuroregulator 104. Typically, the memory 152 includes non-volatile memory. In other embodiments, the memory 152 also can store serial numbers and/or model numbers of the leads 106; serial number, model number, and/or firmware revision number of the external charger 101; and/or a serial number, model number, and/or firmware revision number of the neuroregulator 104.

The CPU 154 of the neuroregulator 104 also may receive input signals and produce output signals to control a signal generation module 159 of the neuroregulator 104. Signal generation timing may be communicated to the CPU 154 from the external charger 101 via the coil 105 and the telemetry module 155. In other embodiments, the signal generation timing may be provided to the CPU 154 from an oscillator module (not shown). The CPU 154 also may receive scheduling signals from a clock, such as 32 KHz real time clock (not shown).

The CPU 154 forwards the timing signals to the signal generation module 159 when therapy signals are to be produced. The CPU 154 also may forward information about the configuration of the electrode arrangement 108 to the signal generation module 159. For example, the CPU 154 can forward information obtained from the external charger 101 via the coil 105 and the telemetry module 155.

The signal generation module 159 provides control signals to an output module 161 to produce therapy signals. In an embodiment, the control signals are based at least in part on the timing signals received from the CPU 154. The control signals also can be based on the electrode configuration information received from the CPU 154.

The output module 161 produces the therapy signals based on the control signals received from the signal generation module 159. In an embodiment, the output module 161 produces the therapy signals by amplifying the control signals. The output module 161 then forwards the therapy signals to the lead arrangement 108.

In an embodiment, the signal generation module 159 receives power via a first power regulator 156. The power regulator 156 regulates the voltage of the power to a predetermined voltage appropriate for driving the signal generation module 159. For example, the power regulator 156 can regulate the voltage in a range of 1-20 volts.

In an embodiment, the output module 161 receives power via a second power regulator 160. The second power regulator 160 may regulate the voltage of the power in response to instructions from the CPU 154 to achieve specified constant voltage levels. The second power regulator 160 also may provide the voltage necessary to deliver constant current to the output module 161.

The output module 161 can measure the voltage of the therapy signals being outputted to the lead arrangement 108 and reports the measured voltage to the CPU 154. A capacitive divider 162 may be provided to scale the voltage measurement to a level compatible with the CPU 154. In another embodiment, the output module 161 can measure the impedance of the lead arrangement 108 to determine whether the leads 106, 106a are in contact with tissue. This impedance measurement also may be reported to the CPU 154. Impedance values of the leads are expected to be about 2000 to 10 megaOhms depending on the material of the electrode or any coating thereon. In embodiments, impedance checks are conducted regularly throughout a treatment period to determine the integrity of the limited conductivity of the electrode. Loss of the limited conductivity of the electrode can result in a larger current leakage across the nerve resulting in nerve damage.

Another embodiment of a circuit is shown in FIG. 3B. The therapy algorithm is divided into a number of very small time segments and the corresponding voltage or current value of that therapy waveform segment is stored into a Field Programmable Gate Array(204). The therapy algorithm voltage or current values may be absolute values or changes relative to the previous voltage or current values. There is an option to retrieve alternate waveforms from an EEPROM (203). The clock oscillator (201) determines the time between successive therapy waveform segments and provides various clock signals for other circuits. The charge pump(205) provides the necessary voltage levels from the battery voltage for operating the circuits, the HV generator (207) and a current source (208) provide the applicable voltage and current levels for the therapy waveform which may be programmable by the user. Various voltage monitors (202), regulators and impedance detectors(206) measure and control the correct operation of the circuits. Some of the functionality is optional such as the memory(203) and telemetry blocks(155).

In addition, the power consumption needs of the neuroregulator 104 can change over time due to differences in activity. For example, the neuroregulator 104 will require less power to transmit data to the external charger 101 or to generate therapy signals than it will need to recharge the internal battery 151.

b. Electrodes

Electrodes, modified electrodes, electrical connections, and electrode coatings impart beneficial features including electrodes and electrode coating materials that are electrically stable over time following implantation in tissue, relatively non-biodegradable yet biocompatible, have high electrical impedance, and limited conductivity. Electrodes or electrode coatings are designed in order to provide sufficient capacitance to create an electrostatic field. In embodiments, the electrodes are employed in blocking of nerve activity upon application of a selected constant voltage or constant current to the nerve with little or no tissue damage.

In embodiments, the electrodes have an impedance of at least about 2000 Ohms or greater, at least about 10,000 Ohms or greater, at least about 60,000 ohms or greater, or at least about 10,000 to 10 megaOhms. The electrode or electrode coating can allow for some field sustaining current and still provide for nerve conduction block or stimulation without creating tissue damage. In embodiments, such field sustaining current is about 400 nC/pulse or less. In embodiments, an electrode or electrode coating is selected that minimizes field sustaining current.

In embodiments, an electrode has an impedance of at least about 2000 to 10 megaOhms, 2000 to 6 megaOhms, 2000 to 1 megaOhm, 2000 to 175,000 Ohms, 2000 to 100000 Ohms, 2000 to 60,000 ohms, or 2000 to 20,000 Ohms. In other embodiments, an electrode has an impedance of at least about 10000 to 10 megaOhms, 10000 to 6 megaOhms, 10000 to 1 megaOhm, 10000 to 175,000 Ohms, 10000 to 100000 Ohms, 10000 to 60,000 ohms, or 10000 to 20,000 Ohms. In yet a further embodiment, an electrode has an impedance of at least about 60,000 to 10 megaOhms, 60,000 to 6 megaOhms, 60,000 to 1 megaOhm, 60,000 to 175,000 Ohms, or 60,000 to 100000 Ohms.

In embodiments, the field sustaining current is about 400 nC/pulse or less, 40 nC/pulse or less, 15 nC/pulse or less, 10 nC/pulse or less, 5 nC/pulse or less, 1 nC/pulse or less, or 0.5 nC/pulse or less.

In embodiments, the high impedance electrode has a resistivity of at least 10² Ohms/cm. In embodiments, the electrodes have a resistivity of about 10² to 10²⁴, 10² to 10²⁰, 10² to 10¹⁵, or 10² to 10¹⁰ ohms/cm. Resistivity's of materials are known to those of skill in the art and as identified, for example, in the Handbook of Polymers. For example, silicon rubber has a resistivity of 4×10¹⁰. Polyurethane has a resistivity of 10¹⁴. Teflon has a resistivity of 10²⁰. High density polyethylene has a resistivity of 10¹⁷.

The present disclosure provides limited conductive coatings which can be deposited on commonly used conductive substrate materials such as platinum, iridium, indium, tin oxide, and tungsten. According to the disclosure, there is provided an implantable electrode having a limited conductive coating comprising acrylic paint, silicone, polyethylene, polystyrene, polyurethane, polyether ether ketone (PEEK), Teflon, polyimide, silica/quartz, iridium oxide, tantalum oxide, aluminum oxide, or parylene. In embodiments, the coating is present in one or more coating layers on a surface thereof, the coating layer or at least one of the coating layers being for contact with body tissue when the electrode is implanted and each coating layer being an electrically non conductive layer of polymer.

Limited conductive coatings can be deposited on the surface of an electrode, for example, by painting it on, hot melt application, sputtering, or photoresist methods. A nonconductive coating is at least about 1 to 1000, 1 to 100, or 1 to 10 microns thick. In embodiments, increasing the thickness of the limited conductive coating on the electrode increases the impedance of the electrode.

The electrodes and leads can have a variety of configurations including bi polar, tripolar and the like. In embodiments, at least two electrodes are found on a single lead. In other embodiments, each lead has one electrode, and multiple leads are employed.

In embodiments, the electrodes are positioned on a target nerve or neural tissue so that an electric field can be created between them. Surface area of the electrode is selected based on the impedance value of the nerve and the charge per pulse to be delivered to the nerve in order to provide downregulation or upregulation of nerve activity. In embodiments, the total charge per pulse delivered to the nerve electrode interface can be modified depending on the surface area of the electrode and the distance that the electrodes are apart. In certain embodiments, the surface area of the electrode is about 0.1 to 20 mm². In embodiments, the distance between the electrodes is about 0.1 mm to 20 mm.

In embodiments a lead contains one or more electrodes. FIG. 15 shows an example distal end of a bipolar lead, such as lead 106 (see FIG. 1). The lead 106 includes a lead body 210 curved to receive a nerve (e.g., a vagus nerve). The lead body 210 contains a high impedance tip electrode 212 configured to contact with the nerve received within the lead body 210. In embodiments, a high impedance tip electrode 212 is capable of delivering an electrical charge to nerves having a diameter ranging from about one millimeter to about four millimeters.

The lead body 210 also can have a suture tab 214 to attach the lead body 210 to the patient's anatomy to stabilize the position of the lead body 210. A first end of a flexible lead extension 216, which encloses a conductor from the electrode 212, couples with the lead body 210. A second, opposite end of the lead extension 216 terminates at a pin connector (not shown) for attachment to a connector (e.g., an IS-1 connector) 122 (shown in FIG. 1).

The lead 106 shown in FIG. 15 also includes a ring electrode 218 surrounding the lead extension 216 at a position spaced from the tip electrode 212. In an embodiment, the surface area of each electrode 212, 218 is greater than or equal to about 0.1 to 20 square millimeters. In embodiments, the surface of the electrode has an impedance of at least 2000 ohms. A suture tab 220 may be provided for placement of the ring electrode 218 on the patient's anatomy in general proximity to the placement of the tip electrode 212 on the nerve.

Another embodiment of a lead for use in the systems described herein is shown in FIG. 14. In this embodiment, the electrodes are embedded as thin strips of conductive material in a nonconductive strip of material. The nonconductive material can be selected from acrylic paint, silicone, polyethylene, polystyrene, or parylene. The surface of the electrodes in contact with the nerve has an impedance of at least 2000 ohms. The lead has at least one turn of a helix with a helix angle that allows for placement of the nerve within the helical turn. The lead also has a suture tab for securing one end of the lead in place.

In yet another embodiment, an electrode configuration is shown in FIG. 16. In this embodiment, the lead body is made of a non conductive material that forms a cuff around the nerve. Along the interior surface of the cuff there are two electrode plates that are located opposite one another. The surface of the plates facing the nerve has an impedance of at least 2000 Ohms.

The high impedance electrodes can be placed in or near any excitable tissue. In embodiments, the device and electrodes described herein can be placed on or near the vagus nerve, cranial nerves, celiac nerve, celiac plexus, renal nerve, splanchnic nerve, glossopharyngeal nerve, or baroreceptors. In embodiments, a target nerve includes the vagus nerve, the splanchnic nerve or the renal nerve.

In embodiments, the electrodes are placed on a vagus nerve, preferably below the diaphragm. The posterior nerve PVN and the anterior AVN are generally on diametrically opposite sides of the esophagus E just below the patient's diaphragm. A first tip electrode 212 of a lead arrangement 108 (FIG. 1) is placed on the anterior vagus nerve AVN. A second electrode 212a of the lead arrangement 108 is placed on the posterior vagus nerve PVN. The electrodes 212, 212a are connected by leads 106, 106a to a neuroregulator 104 (FIG. I).

At the time of placement of the leads 106, 106a, it may be advantageous for the tip electrodes 212, 212a to be individually energized with a stimulation signal selected to impart a neural impulse to cause a detectable physiological response (e.g., the generation of antropyloric waves). The absence of a physiological response may indicate the absence of an overlying relation of the tested electrode 212, 212a to a vagus nerve PVN, AVN. Conversely, the presence of a physiological response may indicate an overlying relation (e.g., correct placement) of the tested electrode 212, 212a to a vagus nerve. After determining the leads 106, 106a create a physiologic response, the electrodes 212, 212a can be attached to the nerves PVN, AVN.

The therapies as previously described could be employed by using blocking electrodes or stimulation electrodes or both in order to down-regulate and/or up-regulate the target nerve.

c. Electrical Signal Parameters and Delivered Charge

An electrical signal can be generated with a constant but selectable voltage, constant but selectable current in the devices described herein. While not meant to limit the scope of the disclosure, it is believed using electrodes with high impedance results in nerve conduction block with less delivered charge than that of a low impedance electrode.

In embodiments, the amount of charge per pulse delivered to a target neural tissue to result in at least a partial downregulation or upregulation of nerve activity can be determined by the impedance of the electrode, the size of the electrode, the distance of the electrodes from one another. A constant voltage or current at a selected frequency is then selected using the following equation:

Capacitance=∈ᵣ∈₀*A/d (1)

where ∈ᵣ=relative static permittivity, ∈₀=electric constant, *=multiplication, A=the area of the electrodes, and d=the distance between electrodes. Pulse width may be adjusted as therapy continues to increase efficacy of the therapy.

For example, for 2 high impedance electrodes with negligible field sustaining current, the current fills the capacitance of the nerve electrode interface. For 2 electrodes, each with an area of 5 mm²′ and a separation of 2 mm, the capacitance would=∈ᵣ∈₀*A/d=(8.854×10⁻¹² F m⁻¹)*3*(5 mm²/2 mm)=66 picoFarad. Since capacitance is defined as charge (in coulombs (C)) divided by electric potential (in volts (V)), then charge=voltage*capacitance. At a selected voltage of 8 volts, the charge/pulse=(8 V)*(66 pF)=0.53 nC to charge the electrode to nerve capacitance. This is a 1,600 fold decrease in charge/pulse to induce a conduction block than is necessary under the same conditions using a low impedance electrode with an impedance of 1000 Ohms or less.

Electrical signal parameters are designed in order to provide for a certain amount of delivered charge/pulse using a high impedance electrode as compared to a typical low impedance electrodes. In embodiments, the impedance of the electrode, the size of the electrode, and the distance of the electrode are determined. As discussed above, in embodiments, impedance can vary from about 2000 Ohms to 10 megaOhms. In embodiments, the size of the electrode can vary from about 0.1 to about 20 mm². In embodiments, the distance between the electrodes can range from about 0.1 to about 20 mm.

In embodiments, frequencies are selected that provide for upregulating and/or down regulating signal. For a downregulating or blocking signal, frequencies are selected of 200 Hz or greater. For example, a frequency of at least about 200 to 10,000 Hz, 200 to 5000 Hz, 200 to 2500 Hz, 200 to 1000 Hz, 250 to 10,000 Hz, 250 to 5000 Hz, 250 to 2500 Hz, 250 to 1000 Hz, 500 to 10,000 Hz, 500 to 5000 Hz, 500 to 2500 Hz, or 500 to 1000 Hz. For an upregulating signal, frequencies are selected at less than 200 Hz. For example, about 1 to 195 Hz, 1 to 150 Hz, 1 to 100 Hz, 1 to 75 Hz, 1 to 50 Hz, or 1 to 25 Hz.

If a high frequency conduction blocking signal (eg 200 Hz or greater) using alternating current is applied to a target nerve using a constant but selectable voltage, the voltage can be selected from about 1 volt to about 50 volts, about 1 volt to 25 volts, about 1 volt to about 15 volts, or about 1 volt to about 10 volts. In embodiments, the voltage is about 8 to 10 volts in order to minimize power requirements of the battery.

If a high frequency conduction blocking signal (eg 200 Hz or greater) using alternating current is applied to a target nerve using constant current, the current can range from about 0.1 to 15000 μAmp, 0.1 to 1 μAmp, about 1 to 10 μAmp, about 10 to 300 μAmp, about 100 to 1000 μAmp, or about 1000 to 15000 μAmp.

If a low frequency upregulating signal (eg. less than 200 Hz) using alternating current is applied to a target nerve using a constant but selectable voltage, the voltage can be selected from about 1 volt to about 50 volts, about 1 volt to 25 volts, about 1 volt to about 15 volts, or about 1 volt to about 10 volts. In embodiments, the voltage is about 8 to 10 volts in order to minimize power requirements of the battery.

If a low frequency upregulating signal (eg. less than 200 Hz) using alternating current is applied to a target nerve using a constant but selectable current, the current can range from about 0.1 to 15000 μAmp, 0.1 to 1 μAmp, about 1 to 10 μAmp, about 10 to 300 μAmp, about 100 to 1000 μAmp, or about 1000 to 15000 μAmp.

In embodiments, the constant voltage or constant current can be generated by an alternating current or direct current source. In embodiments, the constant voltage or constant current can be generated using radiofrequency such that the device does not require a battery as described above.

d. Duty Cycle

In embodiments, the duty cycle can be varied. A duty cycle is defined as the percentage of time current or voltage is delivered in one cycle. In embodiments, a high frequency electrical signal is employed to create a nerve conduction block. In embodiments, the frequency of the signal is 200 Hz or greater, about 200 Hz to about 50,000 Hz, about 200 to 10,000 Hz, about 200 to 5000 Hz, about 200 to 2500 Hz, about 200 to 1000 Hz, about 200 to 500 Hz, about 300 Hz to about 50,000 Hz, about 300 to 10,000 Hz, about 300 to 5000 Hz, about 300 to 2500 Hz, about 300 to 1000 Hz, or about 300 to 500 Hz. In embodiments, the external component is configured to allow a user to select any one of a number of frequencies.

The pulse width of a high frequency electrical signal of the same frequency can be varied to vary the duty cycle from about 1 to 100%. For example, a high frequency signal of 5000 Hz has a 100% duty cycle when the pulse width is 100 microseconds. If the frequency is maintained at 5000 Hz, the duty cycle can be decreased by decreasing the pulse width. For example, a pulse width of 10 microseconds is a 10% duty cycle. It has been shown that pulse widths of a high frequency electrical signal that are less than 100% duty cycle are sufficient to create a nerve conduction block using the limited conductivity electrodes described herein. In embodiments, an external component is configured to provide a selection of duty cycles so that the % of blocking of nerve activity can be adjusted based on efficacy for treatment of the disorder and comfort of the patient.

For application of a low frequency electrical signal in order to upregulate activity on a target neural tissue, the frequency selected is about 200 Hz or less about 0.01 to 150 Hz, about 0.01 to 100 Hz, or about 0.01 to 50 Hz. For example, for a biphasic electrical signal delivered at 50 Hz, a pulse width of 10 millisecond (ms) is a 100% duty cycle. Typical pulse widths range from about 0.06-0.8 ms, about 0.06-1 ms or about 0.4-10 ms.

In embodiments, a therapy cycle can include a duty cycle that starts at 1% and increases to 100% during the on time. During the on time, in the case of a 5000 Hz signal, the pulse width of the electrical signal can be increased incrementally from about 1 microsecond up to 100 microseconds. In other embodiments, the duty cycle begins at 100% and decreases to 1% during an on time. During the on time, in the case of a 5000 Hz signal, the pulse width of the electrical signal can be decreased incrementally from about 100 microseconds to 1 microsecond.

Variation of the pulse width of the electrical signal using the systems described herein provides a method to vary the % of blocking of the nerve activity. For example, a 10 microsecond pulse provides about 10% or less blocking of nerve activity. As the pulse width increases up to 100 microseconds, the blocking activity increases to about 40% or greater. If the original pulse width selected does not provide efficacious therapy for the disorder pulse width may be increased in order to increase the % of nerve activity blocked.

B. System Software

The external charger 101 and the neuroregulator 104 contain software to permit use of the therapy system 100 in a variety of treatment schedules, operational modes, system monitoring and interfaces as will be described herein.

1. Treatment Schedule

To initiate the treatment regimen, the clinician downloads a treatment specification and a therapy schedule from an external computer 107 to the external charger 101. In general, the treatment specification indicates configuration values for the neuroregulator 104. For example, in the case of vagal nerve treatment for obesity, the treatment specification may define the amplitude, fixed but selectable voltage or current, frequency, impedance values of the electrode, and pulse width for the electrical signals emitted by the implanted neuroregulator 104. In another embodiment, “ramp up” time (i.e., the time period during which the electrical signals builds up to a target amplitude) and “ramp down” time (i.e., the time period during which the signals decrease from the target amplitude to about zero) can be specified.

In general, the therapy schedule indicates an episode start time and an episode duration for at least one day of the week. An episode refers to the administration of therapy over a discrete period of time. Preferably, the clinician programs an episode start time and duration for each day of the week. In an embodiment, multiple episodes can be scheduled within a single day. Therapy also can be withheld for one or more days at the determination of the clinician.

During a therapy episode, the neuroregulator 104 completes one or more treatment cycles in which the neuroregulator 104 sequences between an “on” state and an “off” state. For the purposes of this disclosure, a treatment cycle includes a time period during which the neuroregulator 104 continuously emits treatment (i.e., the “on” state) and a time period during which the neuroregulator 104 does not emit treatment (i.e., the “off” state). Typically, each therapy episode includes multiple treatment cycles. The clinician can program the duration of each treatment cycle (e.g., via the clinician computer 107).

When configured in the “on” state, the neuroregulator 104 continuously applies treatment (e.g., emits an electrical signal). The neuroregulator 104 is cycled to an “off” state, in which no signal is emitted by the neuroregulator 104, at intermittent periods to mitigate the chances of triggering a compensatory mechanism by the body. For example, if a continuous signal is applied to a patient's nerve for a sufficient duration, the patient's digestive system eventually can learn to operate autonomously.

The daily schedule includes a timeline indicating the times during the day when the treatment is scheduled to be applied to a patient. Duty cycle lines (dashed lines) extend along the time periods during which treatment is scheduled. For example, a first episode is scheduled between 8 AM and 9 AM. In certain embodiments, the treatment schedules address other details as well. For example, the daily schedule indicates details of the waveform (e.g., ramp-up/ramp-down characteristics) and details of the treatment cycles.

2. Lead Impedance Measurement

Embodiments of the therapy system 100 have the ability to independently measure and record lead impedance values. Lead impedance values outside a predefined range may indicate problems or malfunctions within the therapy system 100. These embodiments of the therapy system 100 allow the physician to measure lead impedance on-demand. The therapy system 100 also enables the physician to periodically measure impedance without initiating a blocking therapy setting. Generally, impedance is measured and stored separately for each channel of each electrode configuration. These measurements may be used to establish a nominal impedance value for each patient by calculating a moving average. In embodiments, impedance values range from about 2000 to 6.0 megaOhms. Any decrease in impedance value could indicate that the limited conductivity of the electrode is decreasing due to wear of any coating. A decrease in impedance value to a predetermined amount would trigger an alarm and result in shut down of the therapy in order to avoid excess field sustaining current on the nerve and potential nerve damage. The nominal impedance and impedance tolerance range can be used for system non-compliance monitoring.

3. External Computer Interface

Programmer software, with which the physician can program treatment configurations and schedules, resides on and is compatible with an external computing device 107 (FIG. 1) that communicates with the external charger 101. In general, application software for the computing device 107 is capable of generating treatment programs stored in a commonly accepted data file format upon demand.

The programming interface of the computing device 107 is designed to enable the physician to interact with the components of the therapy system 100. For example, the programming interface can enable the physician to modify the operational modes (e.g., training mode, treatment mode) of the external charger 101. The programming interface also can facilitate downloading treatment parameters to the external charger 101. The programming interface enables the physician to alter the treatment parameters of the neuroregulator 104, and to schedule treatment episodes via the external charger 101. The programming interface also enables the physician to conduct intra-operative testing amongst the components of the therapy system 100. For example, the physician can initiate a lead impedance test via the programming interface. The physician also can program temporary treatment settings for special physiologic testing. The programming interface also can facilitate conducting diagnostic stimulation at follow-up visits between the patient and the physician.

The programming interface of the computing device 107 also enables the physician to access patient data (e.g., treatments delivered and noted physiological effects of the treatment). For example, the programming interface can enable the physician to access and analyze patient data recorded by the therapy system 100 (e.g., stored in the memory 152 of the neuroregulator 104 and/or the memory 181 of the external charger 101). The physician also can upload the patient data to the external computing device 107 for storage and analysis.

The programming interface also can enable the physician to view system operation information such as non-compliant conditions, system faults, and other operational information (e.g., lead impedance) of the therapy system 100. This operational data also can be uploaded to the external computing device 107 for storage and analysis.

4. Programs

One or more therapy programs can be stored in the memory of the external computer 107. The therapy programs can include a range of predetermined parameters and therapy delivery schedules. For example, each therapy program can specify a selectable current or voltage, a frequency, duty cycle, a charge per pulse, a pulse width, ramp-up rates, ramp-down rates, and an on-off cycle period. In an embodiment, one or more of these parameters can be individually and separately programmed. For example, a constant voltage range of about 1 to 20 volts may be selectable with a default value at 8 or 14 volts. The current can range from about 0.1 to 15000 μAmp, 0.1 to 1 μAmp, about 1 to 10 μAmp, about 10 to 300 μAmp, about 100 to 1000 μAmp, or about 1000 to 15000 μAmp with a default value set at 1000 μAmp. In another example, frequencies can be selected from 200 Hz to 10,000 Hz, with a default value set at 5000 Hz. In yet another example, the pulse width can be selected from 1 to 100 microseconds, with a default value of 90 or 10 microseconds.

In embodiments, a therapy delivery schedule can also be selectable. In embodiments; a range of therapy hours per day are selectable from 1 to 24 hours. In embodiments, the default value can be 6, 9, or 12 hours. In addition, the start time or end time of the therapy schedule is selectable. For example, in the case of hypertension, a start time can begin as early as 4 or 5 am. In another example, a start time can be in the late afternoon or evening in order to accommodate shift work. In that case, a start time can range from 4 pm to about 9 pm.

In use, the physician may select any one of these therapy programs and transmit the selected therapy program to the implanted neuroregulator 104 (e.g., via the external charger 101) for storage in the memory of the neuroregulator 104. The stored therapy program then can control the parameters of the therapy signal delivered to the patient via the neuroregulator 104.

Typically, the default parameter settings of the programs are set at the factory, prior to shipment. However, each of these parameters can be adjusted over a certain range, by the physician, using the computer 100 to produce selectable, customized, therapy programs. Using these selectable, customized therapy programs, the physician can manage the patient's care in an appropriate manner.

For example, when patients require more varied therapies, the neuroregulator 104 can store a therapy program including one or more combinations of multiple therapy modes sequenced throughout the day.

C. External Charger

An embodiment of the external charger 101 can change the amplification level of the transmission signal (e.g., of power and/or data) to facilitate effective transmission at different distances between, and for different relative orientations of, the coils 102, 105. If the level of power received from the external charger 101 varies, or if the power needs of the neuroregulator 104 change, then the external charger 101 can adjust the power level of the transmitted signal dynamically to meet the desired target level for the implanted neuroregulator 104.

Waveforms delivered to the nerve to at least partially block nerve activity are designed and selected to minimize power consumption. Minimizing power consumption of the therapy allows for the use of a smaller battery and/or less recharging sessions.

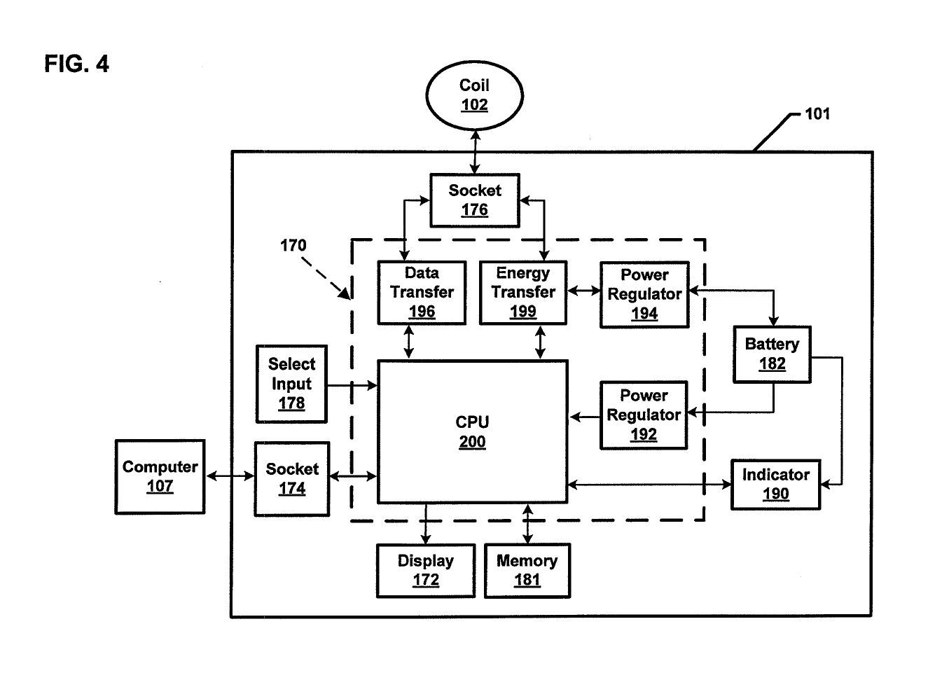

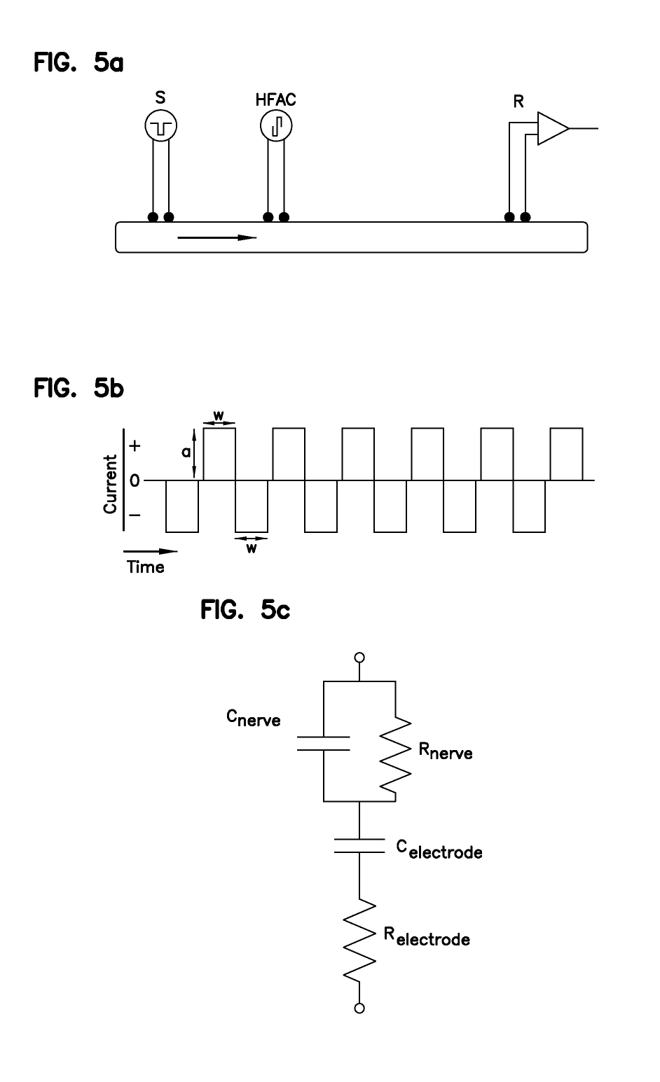

A block diagram view of an example external charger 101 is shown in FIG. 4. The example external charger 101 may cooperate with any of the neuroregulators 104, 104′ discussed above to provide therapy to a patient. The external charger 101 is configured to transmit to the neuroregulator 104 (e.g., via an RF link) desired therapy parameters and treatment schedules and to receive data (e.g., patient data) from the neuroregulator 104. The external charger 101 also is configured to transmit energy to the neuroregulator 104 to power the generation of therapy signals and/or to recharge an internal battery 151 of the neuroregulator 104. The external charger 101 also can communicate with an external computer 107.

In general, the external charger 101 includes power and communications circuitry 170. The power and communications circuitry 170 is configured to accept input from multiple sources, to process the input at a central processing unit (CPU) 200, and to output data and/or energy (e.g., via coil 102, socket 174, or display 172). It will be appreciated that it is well within the skill of one of ordinary skill in the art (having the benefit of the teachings of the present invention) to create such circuit components with such function.

For example, the circuit power and communications circuit 170 can be electrically connected to the external coil 102 for inductive electrical coupling to the coil 105 of the neuroregulator 104. The power and communications circuit 170 also can be coupled to interface components enabling input from the patient or an external computing device (e.g., a personal computer, a laptop, a personal digital assistant, etc.) 107. For example, the external charger 101 can communicate with the computing device 107 via an electrically isolated Serial port.

The external charger 101 also includes a memory or data storage module 181 in which data received from the neuroregulator 104 (e.g., via coil 102 and socket input 176), the external computer 107 (e.g., via socket input 174), and/or the patient (e.g. via select input 178) can be stored. For example, the memory 181 can store one or more parameters. therapy programs and/or therapy schedules provided from the external computer 107. The memory 181 also can store software to operate the external charger 101 (e.g., to connect to the external computer 107, to program external operating parameters, to transmit data/energy to the neuroregulator 104, and/or to upgrades the operations of the CPU 200). Alternatively, the external charger 101 can include firmware to provide these functions. The memory 181 also can store diagnostic information, e.g., software and hardware error conditions.

An external computer or programmer 107 may connect to the communications circuit 170 through the first input 174. In an embodiment, the first input 174 is a port or socket into which a cable coupled to the external computer 107 can be plugged. In other embodiments, however, the first input 174 may include any connection mechanism capable of connecting the external computer 107 to the external charger 101. The external computer 107 provides an interface between the external charger 101 and a physician (e.g., or other medical professional) to enable the physician to program therapies into the external charger 101, to run diagnostic and system tests, and to retrieve data from the external charger 101.

The second input 176 permits the external charger 101 to couple selectively to one of either an external power source 180 or the external coil 102 (see FIG. 1). For example, the second input 176 can define a socket or port into which the power source 180 or external coil 102 can plug. In other embodiments, however, the second input 176 can be configured to couple to a cable or other coupling device via any desired connection mechanism. In one embodiment, the external charger 101 does not simultaneously connect to both the coil 102 and the external power source 180. Accordingly, in such an embodiment, the external power source 180 does not connect directly to the implanted neuroregulator 104.

The external power source 180 can provide power to the external charger 101 via the second input 176 when the external charger 101 is not coupled to the coil 102. In an embodiment, the external power source 180 enables the external charger 101 to process therapy programs and schedules. In another embodiment, the external power source 180 supplies power to enable the external charger 101 to communicate with the external computer 107 (see FIG. 1).

The external charger 101 optionally may include a battery, capacitor, or other storage device 182 (FIG. 4) enclosed within the external charger 101 that can supply power to the CPU 200 (e.g., when the external charger 101 is disconnected from the external power source 180). The power and communications circuit 170 can include a power regulator 192 configured to receive power from the battery 182, to regulate the voltage, and to direct the voltage to the CPU 200. In a preferred embodiment, the power regulator 192 sends a 2.5 volt signal to the CPU 200.

The battery 182 also can supply power to operate the external coil 102 when the coil 102 is coupled to the external charger 101. The battery 182 also can supply power to enable the external charger 101 to communicate with the external computer 107 when the external power source 180 is disconnected from the external charger 101. An indicator 190 may provide a visual or auditory indication of the remaining power in the battery 182 to the user.

In an embodiment, the battery 182 of the external charger 101 is rechargeable. A decrease in charge per pulse of at least 2 to 80000 fold results in a significant energy savings that would allow for use of a smaller battery in a smaller device, or reduced charging of once per month or less. For example, the external power source 180 may couple to the external charger 101 to supply a voltage to the battery 182. In such an embodiment, the external charger 101 then can be disconnected from the external power source 180 and connected to the external coil 102 to transmit power and/or data to the neuroregulator 104.

In an alternative embodiment, the battery 180 is a replaceable, rechargeable battery, which is recharged external to the external charger 101 in its own recharging stand. In yet another embodiment, the battery 182 in the external charger 101 can be a replaceable, non-rechargeable battery.

In use, energy from the external power source 180 flows through the second input 176 to an energy transfer module 199 of the power and communications circuit 170. The energy transfer module 199 directs the energy either to the CPU 200 to power the internal processing of the external charger 101 or to the battery 182. In an embodiment, the energy transfer module 199 first directs the energy to a power regulator 194, which can regulate the voltage of the energy signal before sending the energy to the battery 182.

In some embodiments, the external coil 102 of the external charger 101 can supply energy from the battery 182 to the internal coil 105 of the neuroregulator 104 (e.g., to recharge the internal power source 151 (FIG. 3) of the neuroregulator 104). In such embodiments, the energy transfer module 199 receives power from the battery 182 via the power regulator 194. For example, the power regulator 194 can provide a sufficient voltage to activate the energy transfer module 199. The energy transfer module 199 also can receive instructions from the CPU 200 regarding when to obtain power from the battery 182 and/or when to forward power to the external coil 102. The energy transfer module 199 delivers the energy received from the battery 182 to the coil 102 of the external charger 101 in accordance with the instructions provided by the CPU 200. The energy is sent from the external coil 102 to the internal coil 105 of the neuroregulator 104 via RF signals or any other desired power transfer signal. In an embodiment, therapy delivery at the neuroregulator 104 is suspended and power is delivered from the external charger 101 during recharging of the internal power source 151.

In some embodiments, the external charger 101 controls when the internal battery 151 of the implanted neuroregulator 104 is recharged. In embodiments, the implanted neuroregulator 104 controls when the battery 151 is recharged. These details typically parallel the battery manufacturer's recommendations regarding how to charge the battery.

As noted above, in addition to power transmissions, the external coil 102 also can be configured to receive data from and to transmit programming instructions to the neuroregulator 104 (e.g., via an RF link). A data transfer module 196 may receive and transmit data and instructions between the CPU 200 and the internal coil 105. In an embodiment, the programming instructions include therapy schedules and parameter settings. Further examples of instructions and data transmitted between the external coil 102 and the implanted coil 105 are discussed in greater detail herein.

Example functions capable of selection by the user include device reset, interrogation of battery status, interrogation of coil position, and/or interrogation of lead/tissue impedance. In other embodiments, a user also can select measurement of tissue/lead impedance and/or initiation of a stomach contraction test. Typically, the measurement and testing operations are performed when the patient is located in an operating room, doctor's office, or is otherwise surrounded by medical personnel.

In another embodiment, the user can select one or more parameters, programs and/or therapy schedules to submit to the memory 152 of the neuroregulator 104. For example, the user can cycle through available parameters or programs by repeatedly pressing the selection button 178 on the external charger 101. The user can indicate the user's choice by, e.g., depressing the selector button 178 for a predetermined period of time or pressing the selector button 178 in quick succession within a predetermined period of time.

In use, in some embodiments, the external charger 101 may be configured into one of multiple modes of operation. Each mode of operation can enable the external charger 101 to perform different functions with different limitations. In an embodiment, the external charger 101 can be configured into five modes of operation: an Operating Room mode; a Programming mode; a Therapy Delivery mode; a Charging mode; and a Diagnostic mode.

D. Methods

In another aspect, the disclosure provides methods of using the system described herein. In embodiments, a method of treating a disorder in a subject comprises applying an electrode to a target nerve, wherein the electrode has an impedance of at least 2000 ohms and is operatively coupled to an implantable neuroregulator; applying a therapy cycle to the target nerve, wherein the therapy cycle comprises applying an electrical signal to the electrode intermittently, wherein the electrode signal is applied using a constant voltage or constant current and is selected to downregulate activity on the target nerve. In other embodiments the electrical signal is selected to upregulate activity on the nerve.

Methods of the disclosure can be applied to any excitable tissue. In embodiments, a nerve such as the vagus nerve, splanchnic nerve, celiac nerve, celiac plexus, renal nerve, cranial nerves, glossopharyngeal nerve, or baroreceptors are targeted. Disorders for which modulation of nerve activity is desired are selected. Such disorders include obesity, diabetes, hypertension, inflammatory bowel disease, metabolic disorders, pancreatitis, and bulimia.

In embodiments at least two electrodes are applied to a target nerve in order to generate an electrical field. The at least two electrodes can be present in a single or multiple leads. The surface of the electrode contacting the nerve has high impedance. Such electrodes can be obtained by applying one or more coatings that have limited conductivity as described herein. In embodiments, the electrodes have an impedance of at least 2000 ohms as described previously herein.

Application of a therapy cycle involves applying an electrical signal to the nerve via the electrodes. In embodiments, an electrical signal is generated using constant voltage. A constant voltage can be selected and set by the physician ranging from 1 to 50 volts, 1 to 40 volts, 1 to 30 volts, 1 to 20 volts, or 1-10 volts.

The current can range from about 0.1 to 15000 μAmp, 0.1 to 1 μAmp, about 1 to 10 μAmp, about 10 to 300 μAmp, about 100 to 1000 μAmp, or about 1000 to 15000 μAmp.

The constant voltage may be set based on the selected pulse width. For a particular frequency, pulse width can be selected to include a duty cycle of about 1-100%. For example, for an electrical signal of 5000 Hz, a 100% duty cycle will have a pulse width of 100 microseconds. The pulse width can range from 10 to 100 microseconds. The pulse width may be varied during treatment in order to enhance the efficacy of the therapy cycle or in response to the comfort of the patient.

For downregulating activity of a nerve such as the vagus nerve, the frequencies include 200 Hz or greater, about 200 Hz to about 50,000 Hz, about 200 to 10,000 Hz, about 200 to 5000 Hz, about 200 to 2500 Hz, about 200 to 1000 Hz, about 200 to 500 Hz, about 300 Hz to about 50,000 Hz, about 300 to 10,000 Hz, about 300 to 5000 Hz, about 300 to 2500 Hz, about 300 to 1000 Hz, or about 300 to 500 Hz. For an upregulating signal, frequencies are selected at less than 200 Hz. For example, about 1 to 195 Hz, 1 to 150 Hz, 1 to 100 Hz, 1 to 75 Hz, 1 to 50 Hz, or 1 to 25 Hz.

In embodiments, a method of setting the parameters for a therapy cycle comprises selecting a frequency, followed by selecting one or more pulse widths, and then selecting a constant voltage or constant current based on the selected pulse widths. In embodiments, the physician programmer or the external component has a user interface that allow selection of each of these parameters.

### EXAMPLES

Stimulation of neural tissue using low impedance electrodes is typically achieved using charge balanced biphasic current pulses to minimize the generation of direct current and the production of harmful electrochemical products. The extent to which current affects the nerve can be modeled using a simplified electrode system as shown in FIG. 5C. The figure shows the nerve to electrode interface. In this system, the electrode to nerve capacitance is generally high (in the order of tens to hundreds of pF), while the resistance is low (in the order of tens of Ohms).

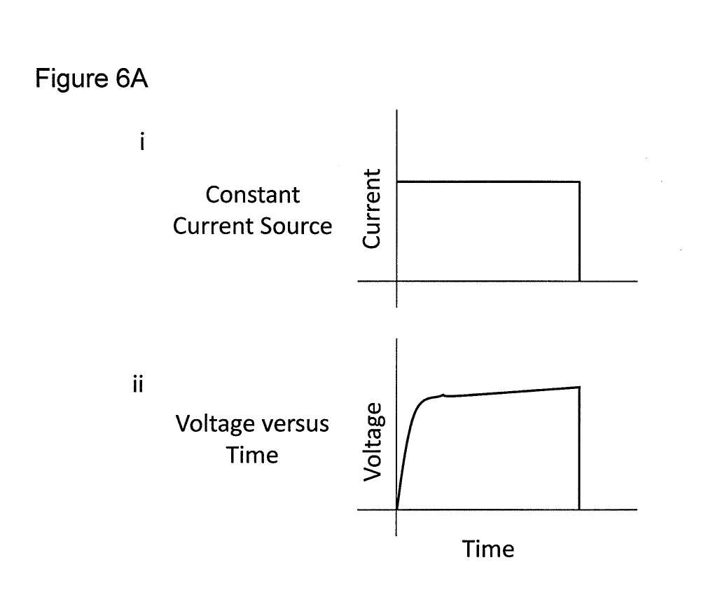

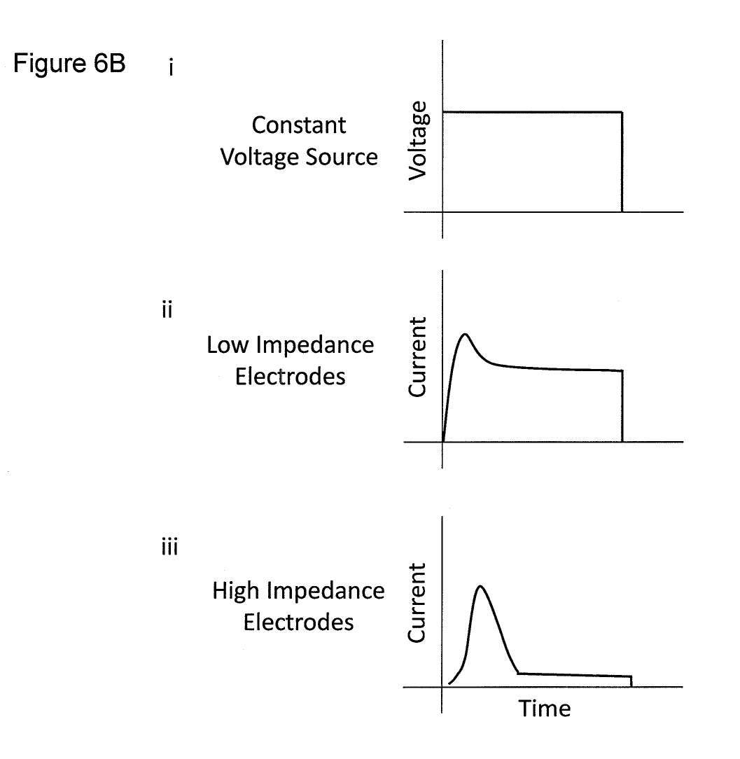

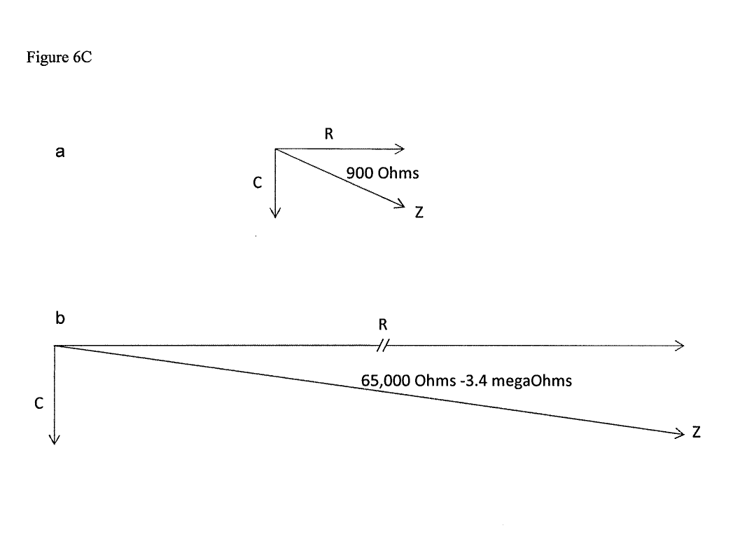

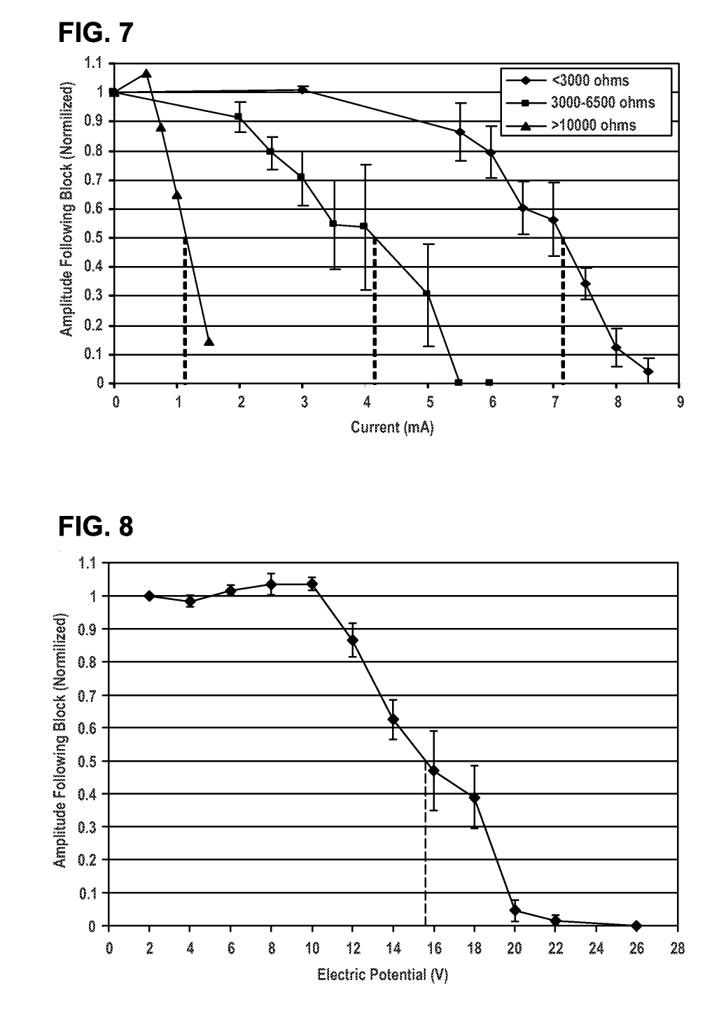

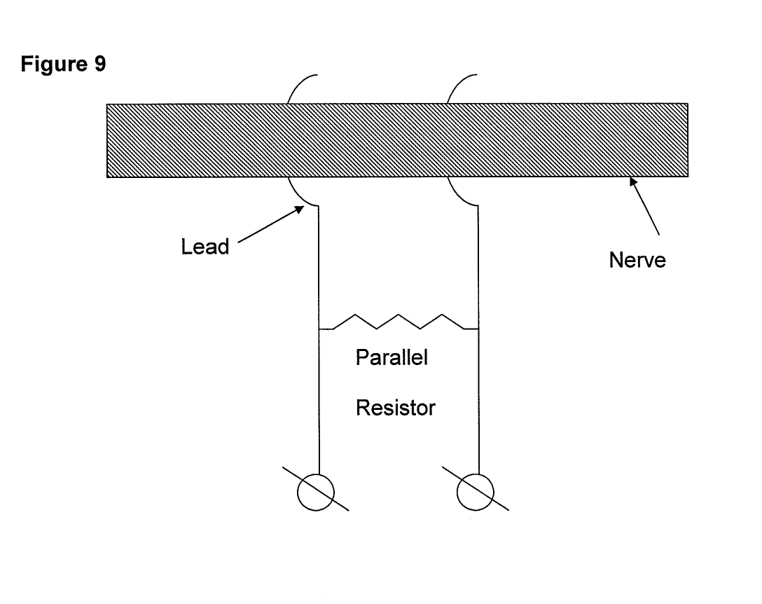

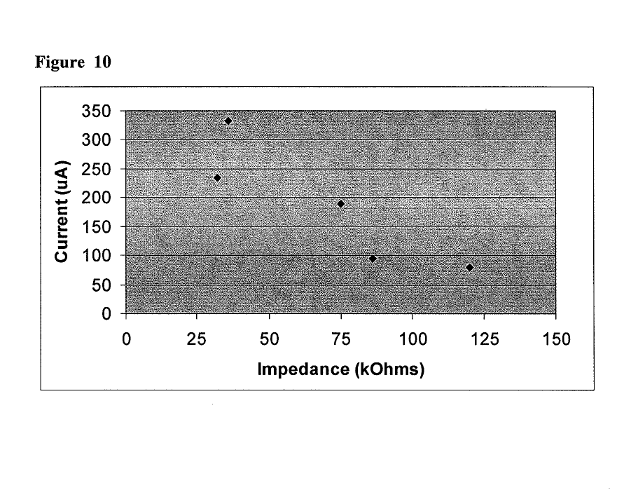

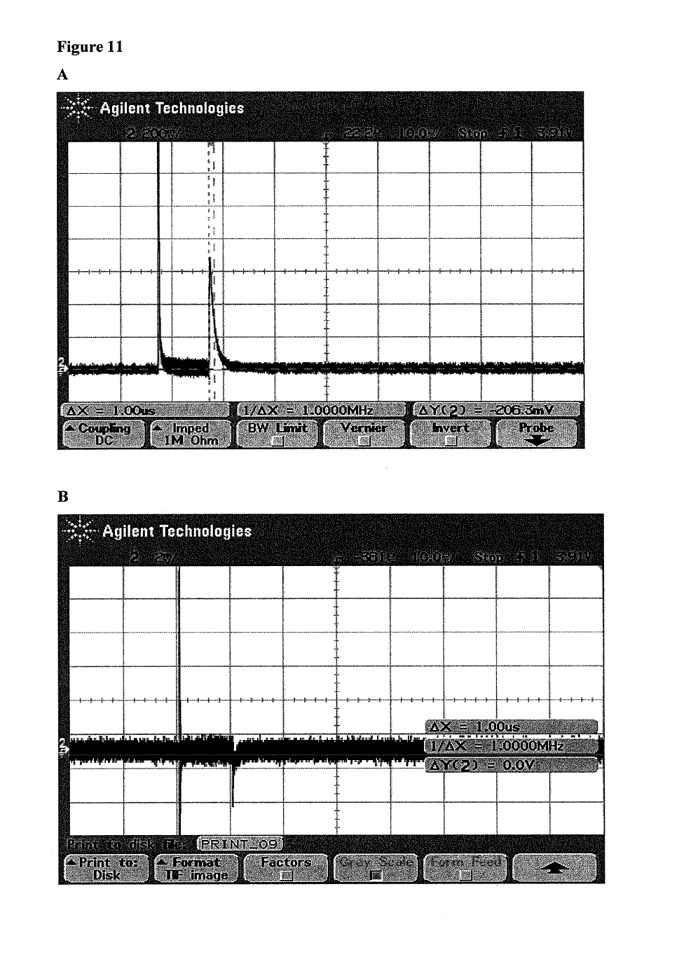

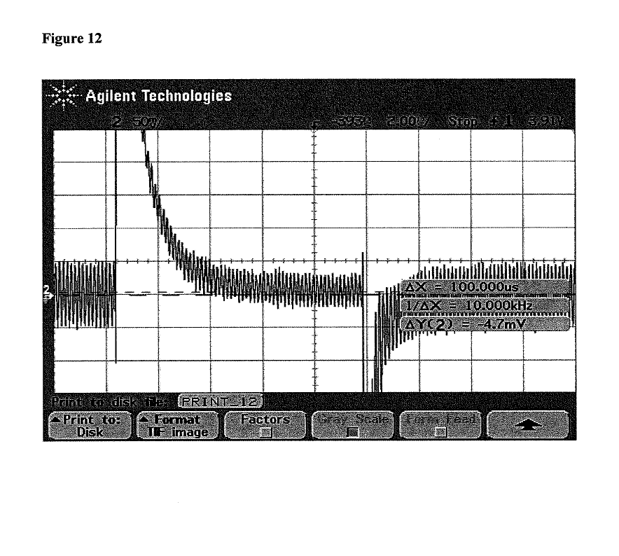

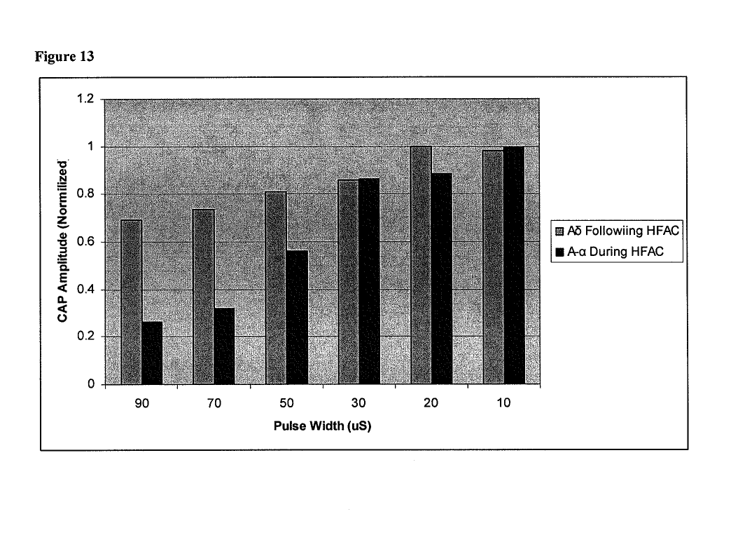

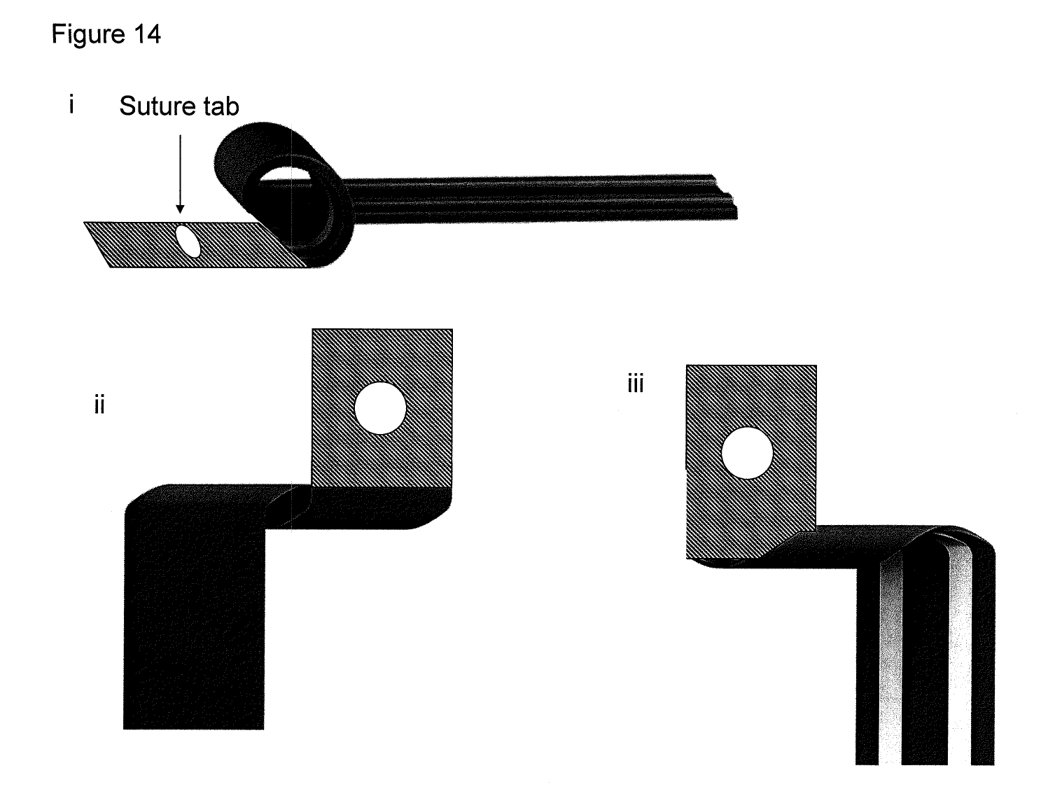

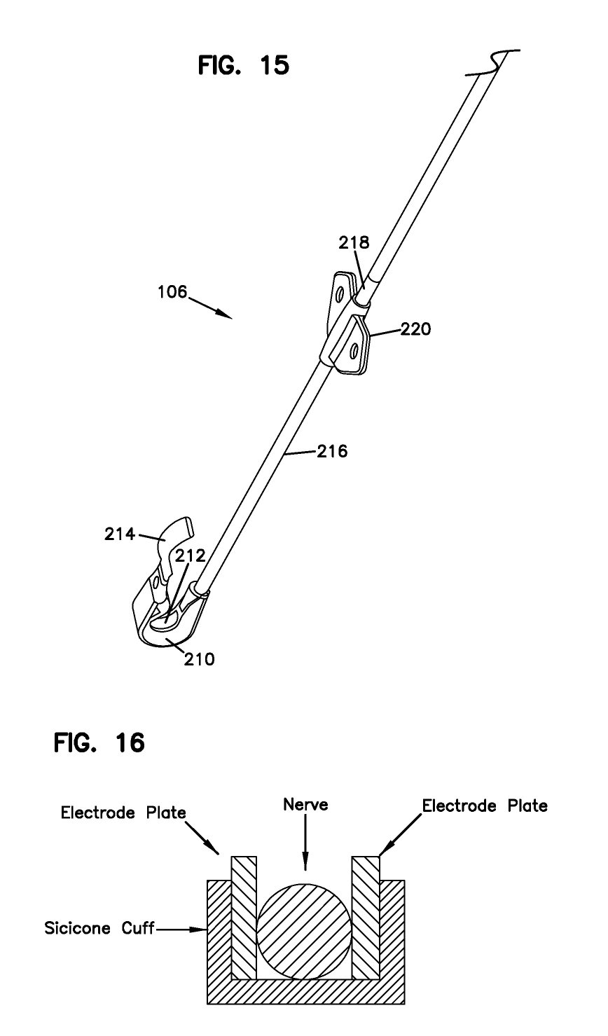

In a current regulated device, the voltage across low impedance electrodes will quickly rise due to current flowing across the impedance of the nerve membrane. With time, the voltage will keep rising, albeit at a slower rate, due to charge filling the electrode to nerve capacitance. (See FIG. 6A (i and ii)). In a constant voltage regulated device, there is an initial current spike due to charging the capacitance of the nerve and electrode system. FIG. 6B (ii and iii). The remaining current will be essentially determined by the parallel resistance of the nerve. In the case of a system with a typical low impedance electrode, the current is maintained at a higher level by passage of the current through the nerve.

While not meant to limit the disclosure, it is thought that placing a voltage or current signal on electrodes on or near a nerve, leads to the formation of an electric field that influences the ion gates in the nerve, and in the case of a high frequency signal this results in a down regulation of nerve activity. It is believed that charging the capacitance of the electrode initiates this electrical field and that continued current flow through the electrode maintains this field. The capacitance of conventional low impedance electrode is a function of the area of the electrode to nerve interface.

By adding a high impedance dielectric coating to the electrode, the capacitance of the electrode will increase equivalent to the dielectric constant of the coating which is typically in the order of 2 to 4 times higher than a conventional low impedance electrode. The resistance of the electrode nerve interface will increase more significantly and can be in the order of 10,000 to 1,000,000 times higher than a conventional low impedance electrode. Applying a voltage or current signal on high impedance electrodes will result in the initiation of an electric field as soon as the electrode capacitance is charged, and because the high impedance dielectric coating on the electrode prevents the charge from dissipating, this field can be maintained at lower currents than in conventional electrodes. See FIG. 6B(iii). Rapid charging of the capacitance of the electrode using an optimal voltage or current and careful matching of the electrode impedance to the nerve and its environment in the body allows for significant reduction in charge required to influence ion gates in nerves. In addition, high impedance electrodes have increased safety profile due to a decrease in the charge/pulse delivered to the nerve.

To illustrate the charge reduction using a high impedance electrode the nerve capacitance is modeled with a simplified model. FIG. 5C. We estimate that the capacitance of an electrode=∈ᵣ∈₀*A/d where ∈ᵣ=relative static permittivity, ∈₀=electric constant, A=the area of the electrodes, and d=the distance between electrodes. In the case of the traditional low impedance electrode ∈ᵣ is approximately 1. With an electrode surface area of 5 square millimeters (mm²) and separation of 2 mm, the capacitance=∈ᵣ∈₀*A/d=(8.854×10⁻¹² F m⁻¹)*1*(5 mm²/2 mm)=22 pFarads (pF). The charge on the low impedance electrode at a stimulation voltage of 8 V equals voltage*capacitance=(8V)*(22 pF)=0.18 nC. The resistive aspect of the traditional low impedance electrode is modeled at approximately 1000 Ohms. Ohms law can be used to approximate the amount of current necessary to sustain the electric field. This current equals voltage/resistance=4.6 V/1000 Ohms=0.0046 amps. At 5000 Hz, the pulse width for a biphasic pulse is (1/5000 Hz)/2=0.0001 seconds. Since charge=pulse width*current, the charge necessary to sustain the electric field equals 0.0046 amps*0.0001 seconds=460 nC.

By coating electrodes with a limited conducting material, the impedance increases significantly, for example, ranging from 2000 ohms to 10 megaOhms (FIG. 6Cii). When the surface area and distance between the high and low impedance electrodes remains fixed, the capacitance only changes by ∈ᵣ for the material applied to the electrode. In one embodiment, the capacitance increases by approximately 3 times to 66 pF. The charge on the electrode at a stimulation voltage of 8 V equals voltage*capacitance=(8V)*(66 pF)=0.53 nC. Assuming the high impedance electrode is approximately 100,000 Ohms, the current to sustain the electric field equals voltage/resistance=8 V/100,000 Ohms=0.00008 amps. At 5000 Hz, the pulse width for a biphasic pulse is (1/5000 Hz)/2=0.0001 seconds. Since charge=pulse width*current, the charge necessary to sustain the electric field equals 0.00008 amps*0.0001 seconds=8 nC. This is a decrease of about 60 times in the charge/pulse necessary to induce a conduction block under the same conditions as compared to a low impedance electrode with an impedance of 1000 Ohms or less.

The decrease in the amount of charge required to achieve nerve conduction downregulation and/or upregulation can be determined by selecting a current or voltage, electrode area and then selecting the appropriate coating and thickness to achieve a high impedance value. Increasing the impedance of the electrode aims to reduce the current necessary to sustain the electrical field to the lowest value that allows down or up regulation of the nerve. Charge per pulse can be calculated for electrodes of differing impedance values.

Table 1 summarizes the calculated charge/pulse with different impedance electrodes.

TABLE 1
Summary of calculated charge/pulse using various impedance electrodes.
The voltage is 8 V, the frequency is 5000 Hz, the electrode surface
area is 5 mm² and the separation is 2 mm.

| Impedance | Sustaining charge/ | Capacitive charge/ | Total charge/ |
|---|---|---|---|
| (kOhm) | pulse (nC) | pulse (nC) | pulse (nC) |
| 1 (low | 800.00 | 0.53 | 800.53 |
| impedance) | | | |
| 2 | 400.00 | 0.53 | 400.53 |
| 20 | 40.00 | 0.53 | 40.53 |
| 65 | 12.31 | 0.53 | 12.84 |
| 100 | 8.00 | 0.53 | 8.53 |
| 175 | 4.57 | 0.53 | 5.10 |
| 1000 | 0.80 | 0.53 | 1.33 |
| 5000 | 0.16 | 0.53 | 0.69 |

Note:
as the impedance increases the sustaining charge/pulse approaches the capacitive charge/pulse.

### Example 1

In terms of high frequency conduction block with low impedance electrodes the energy requirements can be viewed as the charge/pulse. When the current amplitude and pulse width are known the charge/pulse can be calculated. For example, when vagus nerve Aδ waves are 50% blocked following a 5000 Hz signal at an approximate current of 2.5 mA and a pulse width of 90 μs (Waataja et al., 2011), charge per pulse under those conditions is calculated. Since charge/pulse=current*pulse width the charge/pulse to approximately block 50% of the vagus nerve Aδ wave is 2.5 mA*of 90 μs=225 nC. An approximate 50% block of vagus nerve C waves following a 5000 Hz signal with a 90 μs pulse width requires approximately 7.25 mA (Waataja et al., 2011). Thus, for conduction block of the C wave the charge/pulse=7.25 mA*90 μs=653 nC.

We examined a method to block conduction through the vagus nerve with a considerably lower amount of energy. The method involves using high impedance electrodes to limit the current required to sustain an electric field (FIG. 6B(iii). The present study was designed to determine if, and to what extent, voltage, electrode/nerve impedance, capacitance and current required to sustain the electric field play in conduction block through the vagus nerve.

We used an isolated rat vagus nerve preparation to test the effects of 5000 Hz HFAC, at different impedances and voltages, on electrically evoked compound action potentials (CAPs).

### Methods

### Vagus Nerve Isolation

Experiments were approved by the Institutional Animal Care and Use Committee at the University of Minnesota and performed on adult male Sprague-Dawley rats (225-375 g, n=10). Rats were killed with an overdose of isoflurane. An incision was made just below the sternum to expose the rib cage. The ribcage was then removed to expose the thoracic and cervical vagus. At this point, oxygen-saturated synthetic interstitial fluid (SIF (Koltzenburg et al., 1997), (in mM) NaCl 108, KCl 3.5, CaCl2 1.5, MgSO4 0.7, NaHCO3 26, NaH2PO4 1.7, sodium gluconate 9.6, glucose 5.5 and sucrose 7.6) was introduced to the exposed thoracic and cervical cavities. The left and right vagus nerves were located at level of the carotid bifurcation and gently dissected away from the rat towards the heart. The nerve was further dissected to remove excess tissue, vasculature and fat. After the nerve was isolated it was placed in ice-cold oxygenated SIF.

### Electrophysiology

Excised nerves were suspended on 3 sets of bipolar hook electrodes in mineral oil at 36° C. The electrode arrangement is shown in FIG. 5a. The stimulation and recording electrodes included pairs of platinum/iridium and Ag/AgCl wire (0.01-0.015 inch diameter), respectively. The electrode delivering HFAC was a pair of platinum-iridium ribbon wires (0.02 inch thickness; 0.05 inch width) separated by 2 mm. In some experiments the platinum-iridium ribbon wires were covered with an acrylic-based paint, silicon or parylene. The stimulating and HFAC electrodes were typically located at the level of the cervical vagus, and recording electrodes were situated at the thoracic end. A layer of SIF under the mineral oil provided a grounding path.

The vagus nerve was activated through the stimulation electrodes with monophasic (negative) pulses (0.1 to 10 msec duration) generated by an electrical stimulator (Model A300, World Precision Instruments, Sarasota, Fla., USA) and delivered at 0.5-1 Hz through a constant-current stimulus isolation unit (10 mA maximum, WPI model A360).

Stimulus-evoked nerve signals were led from recording electrodes to the headstage of a differential amplifier (WPI model DAM 80, 1000× gain, typical bandpass of 10 Hz to 3 kHz) and referenced to a Ag/AgCl pellet in the underlying SIF. Interference from line noise was minimized with a signal conditioning device (Humbug, Quest Scientific, North Vancouver, BC, Canada) before the signal was led in parallel to oscilloscopes and a data acquisition system (Power 1401 with Spike 2, Cambridge Electronic Design, Cambridge, England).

### HFAC

High Frequency Alternating Current was generated by a proprietary (EnteroMedics, Inc. St. Paul, Minn. USA) computer-controlled device. In some experiments, applications of HFAC included charge-balanced alternating biphasic current pulses (90 to 10 μs duration) delivered at 5000 Hz for 1 minute (FIG. 5B). In each experiment, different HFAC current amplitudes were delivered in random order.

In some experiments a constant voltage source was used. To create a constant voltage source, with a current controlled device, a resistor was placed in parallel with the nerve (FIG. 9). Thus, the voltage drop across the resistor and the nerve would be equal. Using Ohms law (voltage=current*resistance) a determined voltage could be applied across the nerve by applying a given current.

### Measurements and Analyses

Isolated vagus nerves were electrically activated at a cervical location, and the conducted activity was recorded as compound action potential waveforms from the thoracic end. Conduction distance was measured between nearest stimulation and recording electrodes, latency was measured from onset of stimulus artefact to time of maximal peak negativity of CAP waveforms, and peak conduction velocity of each waveform was estimated as distance/latency (m/s). Peak waveform negativity was taken as a measure of waveform amplitude.

Before testing the effects of HFAC, CAP waveforms were first optimized by adjusting stimulus duration and amplitude. Typically, CAP waveform amplitudes at 1.5 to 2.0× stimulus threshold were established as baseline measures. CAP amplitudes were measured continuously for at least 10 minutes before HFAC (baseline), immediately after HFAC, at 30 seconds following HFAC and every subsequent minute following HFAC, until recovery was evident. CAP amplitudes after HFAC were expressed as a ratio relative to baseline values. Full recovery of CAP waveforms was considered to be 95% of baseline amplitude. When comparing groups of nerves, baseline measures of CAP amplitudes were normalized. HFAC intensity (mA or volts) was varied while keeping frequency, waveform timing, and duration (1 minute) constant. Following any full blockade of CAP waveforms, HFAC at higher current or voltage amplitudes was not tested.

Curve fitting, statistical analyses, and graphing were performed with SigmaPlot/SigmaStat (Systat Software, Chicago, Ill., USA) and Microsoft Excel (Microsoft, Redmond Wash., USA). All data are presented as mean±SEM, and a P level of 0.05 was used in tests of significance.

### Results

### High Frequency Conduction Block Through the Vagus Nerve is Voltage Dependent

High frequency induced conduction block through the vagus nerve using conventional low impedance electrodes was tested on evoked C-waves (conduction velocity<1 m/s) using current amplitudes from 0.5 to 8.5 mA. Over a single nerve there was a clear relationship between current amplitude and C-wave attenuation. However, there was no clear relationship between current amplitude and C-wave attenuation between nerves. For example, in one nerve the C-wave would be abolished at 1.5 mA, however, for another nerve 1.5 mA had no effect. In the other nerve it took 8.5 mA abolish the C-wave.

One of the greatest variables between nerves was differences in impedances. The impedances between HFAC electrodes ranged from 1800 to 19,000 ohms (mean=6500±1100 ohms, n=25 nerves, 12 rats). It was hypothesized that the differences in current amplitude required to achieve block between nerves was due to differences in impedance. Different impedance values between nerves may be due to differences in connective tissue on the nerves. Thus, an impedance test was taken before blocking runs and nerves were grouped into 3 impedance categories; those below 3000 ohms, those between 3000 and 6500 ohms and those greater than 10,000 ohms.

A randomized blocking order of current amplitudes was then created based on the impedance. Lower currents (0.5-1.5 mA) were applied to nerves with high impedance (>10,000 Ohms) and high currents (5.5-8.5 mA) were applied to nerves with low impedances (<3000 Ohms). Nerves that had had impedances that fell in the middle (3000-6500 ohms) had applied currents between 2-5.5 mA. FIG. 7 demonstrates there was a relationship between current amplitude and reduced CAP amplitude following HFAC when nerves were grouped by impedance. The effective current to attenuate 50% of the C-wave for impedances less than 3000 ohms was ˜7.1 mA, for impedances between 3000-6500 ohms the effective current to attenuate 50% of the C-wave was ˜4.2 mA and for impedances greater than 10,000 ohms the effective current to attenuate 50% of the C-wave was ˜1.1 mA.

Unlike current, nerves did not have to be grouped into different categories to determine the amount of voltage required to block evoked vagal C-waves. Grouping all nerves together established a relationship between the voltage required to achieve different magnitudes of C-wave attenuation. The effective voltage to attenuate 50% of the vagus nerve C-wave was ˜15.6 V (FIG. 8).

### Blocking Conduction Through the Vagus Nerve Using High Impedance Electrodes

It required less current amplitude to block conduction through the vagus nerve when impedances were higher. For example, an impedance of 10000 Ohms resulted in a 50% block at 1 mA as compare to an impedance of less than 3000 Ohms which required about 7 mA to generate a 50% block. See FIG. 7. Lower current amplitude would decrease the total amount of energy required to block. Increasing the electrode impedance by coating with a limited-conductive material would decrease the current required to sustain the electric field. Since it was also shown in FIG. 8 that conduction block through the vagus nerve was dependent on voltage, a constant voltage device would be more appropriate to use than a constant current device. Thus, the electrodes were coated with limited-conductive materials to increase impedance and a constant voltage source was created from a constant current device (FIG. 9).

Insulated electrodes were created by coating platinum-iridium ribbon wire with a non-conducting acrylic-based paint. To determine the small amount of current required to sustain the electric field, impedance between the HFAC electrodes was calculated. This was done by using the equation:

Rₑ=(Rₛ*Rₜ)/(Rₛ−Rₜ). (2)

Where Rₛ is the resistance of the parallel resistor, Rₜ is the measured total resistance of the circuit and Rₑ is the resistance between the HFAC electrodes.

A total of 5 nerves were tested with Rₑ impedances ranging from 32 to 120 kOhms. The current required to sustain the electric field could then be calculated by solving Ohms law for current (I=V/Rₑ). The current required to block >50% of the evoked Aδ-wave was between 80 and 333 μA. It should be noted that the current to block ˜50% of Aδ-waves without the acrylic-based coating is between 2,000 and 3,000 μA (Waataja et al. 2011). It should also be noted that with higher impedances it took less current to induce conduction block (FIG. 10). Since charge/pulse is directly proportional to current, then less charge/pulse was required to block with higher impedances.

In order to achieve a more accurate measurement of field sustaining current a resistor was added in series between one of the HFAC electrodes and the current regulator. The sustained voltage was then probed across the series resistor and current calculated by Ohms law. This time, three different coatings were used to increase impedances; silicone, parylene and the acrylic-based paint. The sustained current flowing through the series resistor to induce a 50% block ranged from 22-41 μA (Table 2).

TABLE 2
Impedance and sustained current flowing across the
HFAC electrodes using various electrode masks.

| Material | Electrode Impedance (kOhms) | Sustained Current (μA) |
|---|---|---|
| Silicone | 63 | 23 |
| Parylene (1.2 μM) | 94 | 41 |
| Acrylic | 32 | 22 |

In a different set of experiments higher impedance parylene coated electrodes were used to induce conduction block. This was achieved by using a thicker parylene coating. To obtain an impedance of 250 kOhm, a 5 μM thick parylene coating was used. To obtain an impedance of 5400 and 5800 kOhm an 8 μM thick parylene coating was used. A probe was also used to measure the capacitance at 5000 Hz. Using a selected voltage, measured impedance and measured capacitance the total charge/pulse could be calculated and compared to low impedance electrodes.

These experiments demonstrated 3 major points. First, the same degree of conduction block can be achieved using the high impedance 5 μM and 8 μM parylene coated electrodes as with the low impedance electrodes. Second, when inducing the same degree of block the total charge/pulse was significantly lower with the high impedance electrodes versus the low impedance electrodes. Third, the measured capacitance for the high impedance electrodes (average=95 pF) was similar to that calculated (66 pF) using a parallel plate capacitor for the electrode/nerve interface. These results are summarized in table 3.

TABLE 3
Summary of various percent blocks and total
charge/pulse for different impedance electrodes.

| | | | | Sustained | | Capacitive | Total |
|---|---|---|---|---|---|---|---|
| | % | Impedance | | charge/pulse | Capacitance | charge/pulse | charge/pulse |
| Condition | Block | (kOhm) | Voltage | (nC) | (pF) | (nC) | (nC) |
| Low | 50 | 1 | 2.5 | 225 | NA | NA | 225 |
| Impedance | | | | | | | |
| High | 48 | 250 | 12.4 | 4.5 | 159 | 1.4 | 5.9 |
| Impedance | | | | | | | |
| Low | 10 | 1 | 1 | 90 | NA | NA | 90 |
| Impedance | | | | | | | |
| High | 14 | 5400 | 6.3 | 0.11 | 87 | 0.46 | 0.57 |
| Impedance | | | | | | | |
| High | 12 | 5800 | 12.8 | 0.20 | 39 | 0.23 | 0.43 |
| Impedance | | | | | | | |

Note:
that with the higher impedance electrodes the measured capacitive charge/pulse is close to the sustained charge/pulse.

### Series Resistor Scope Plots During Block

Scope plots of voltage versus time were recorded across the series resistor during block. In this case the 5 μM parylene coated electrode was used to deliver the 5000 Hz blocking signal. As shown in FIG. 11A, the first peak was a result of the device shorting out to assure there was no DC offset. The second spike was due to current charging the capacitance of the 5 μM parylene masked electrode. Note that following the second spike the voltage dropped to nearly zero indicating negligible field sustaining current flowing through the series resistor, i.e. the nerve. With this preparation the A∂-wave was attenuated by 47% at 8.4 volts across the HFAC electrodes.

As shown in FIG. 11 B, using a current probing device in place of the series resistor, the first peak was again a result of the device shorting out to assure there was no DC offset. The second spike was due to current charging the capacitance of the 5 μM parylene masked electrode. Note that following the second spike the current dropped to nearly zero. Thus, the current flowing to the masked electrodes charged the nerve to electrode capacitance with negligible current used to sustain the electric field. The capacitance of the system was measured to be 65 pF. These results were replicated on separate preparations.

Since charging the capacitance of the masked HFAC electrode was significantly shorter than 90 μS (FIG. 12), a 10 μS pulse width was tested. The first spike was due to current charging the capacitance of the 5 μM parylene masked electrode. The second spike was due to current charging the capacitance of electrode in the opposite direction. Note that following the first spike the voltage dropped to practically zero indicating negligible field sustaining current flowing through the nerve. The voltage across the masked HFAC electrode was 8.4 volts and 28% of the Aδ-wave was attenuated.

Testing at Different Pulse Widths

The effect of HFAC induced conduction block through the vagus nerve was tested at different pulse widths while keeping frequency and amplitude fixed using the 5 μM parylene coated electrode (FIG. 5b). A 5000 Hz alternating current signal at 14.2 V_{b-p} (voltage measured from the base to the peak of the waveform) was applied for 1 min. The field sustaining current was negligible. At a 90 μS pulse width, the evoked Aδ-wave was attenuated by 31% following 5000 Hz. This attenuation decreased with shorter pulse widths (FIG. 13).

Faster Aα-waves were also analyzed during the application of 5000 Hz. At a 90 μS pulse width the wave was attenuated by 75%. The attenuation also decreased with shorted pulse widths in a similar fashion to the Aδ-wave (FIG. 13). Thus, the degree of conduction block can be adjusted by changing pulse width while keeping all other variables fixed. This is a novel method to adjust the degree of block through the vagus nerve.

### SUMMARY

These results have demonstrated that impedance can be increased by coating typical low impedance electrodes with a non-conducting material. The coated high impedance electrodes were able to block conduction through nerve by delivering a high frequency electrical signal. Furthermore, the same block could be achieved with significantly less charge/pulse when high impedance electrodes were used versus low impedance electrodes. Decreasing the charge/pulse decreases the energy required to block.

Modifications and equivalents of disclosed concepts such as those which might readily occur to one skilled in the art are intended to be included in the scope of the claims which are appended hereto. In addition, this disclosure contemplates application of a combination of electrical signal treatment by placement of electrodes on one or more nerves. This disclosure contemplates application of a therapy program to down regulate neural activity by application of an electrical signal treatment by placement of electrodes on one or more nerves. This disclosure contemplates application of a therapy program to up regulate neural activity by application of electrical signal treatment by placement of electrodes on one or more nerves. Any publications referred to herein are hereby incorporated by reference.

### REFERENCES

Asala S A, Bower A J 1986 An electron microscope study of vagus nerve composition in the ferret. Anat Embryol (Berl) 175 247-253.
Bhadra N, Kilgore K L 2005 High-frequency electrical conduction block of mammalian peripheral motor nerve. Muscle Nerve 32 782-790.
Davenport H A, Chor H, Dolkart R E 1937 The ratio of myelinated to unmyelinated fibers in regenerated sciatic nerves of Macacus rhesus. J Comp Neurol 67 483-491.
Deurloo K E, Holsheimer J 2003 Fascicular selectivity in transverse stimulation with a nerve cuff electrode: a theoretical approach. Neuromodulation 6 258-269.
Docherty R J, Charlesworth G, Farrag K, Bhattacharjee A, Costa S 2005 The use of the rat isolated vagus nerve for functional measurements of the effect of drugs in vitro. J Pharmacol Toxicol Methods 51 235-242.
Dragstedt L R 1945 Vagotomy for gastroduodenal ulcer. Ann Surg 122 973-989.
Farrag K J, Costa S K, Docherty R J 2002 Differential sensitivity to tetrodotoxin and lack of effect of prostaglandin E2 on the pharmacology and physiology of propagated action potentials. Br J Pharmacol 135 1449-1456.
Feirabend H K, Choufoer H, Ploeger S, Holsheimer J, van Gool J D 2002 Morphometry of human superficial dorsal and dorsolateral column fibres: significance to spinal cord stimulation. Brain 125 1137-1149.
Hulsebosch C E, Coggeshall R E 1982 An analysis of the axon populations in the nerves to the pelvic viscera in the rat. J Comp Neurol 211 1-10.
Kang Y M, Bielefeldt K, Gebhart G F 2004 Sensitization of mechanosensitive gastric vagal afferent fibers in the rat by thermal and chemical stimuli and gastric ulcers. J Neurophysiol 91 1981-1989.
Kilgore K L, Bhadra N 2004 Nerve conduction block utilising high-frequency alternating current. Med Biol Eng Comput 42 394-406.
Koltzenburg M, Stucky C L, Lewin G R 1997 Receptive properties of mouse sensory neurons innervating hairy skin. J Neurophysiol 78 1841-1850.
Kral J G, Gortz L 1981 Truncal vagotomy in morbid obesity. Int J Obes 5 431-435.
Mogyoros I, Kiernan M C, Burke D 1996 Strength-duration properties of human peripheral nerve. Brain 119 439-447
Moore C L, White R H 1996 Sex differences in sensory and motor branches of the pudendal nerve of the rat. Horm Behav 30 590-599.
Ozaki N, Sengupta J N, Gebhart G F 1999 Mechanosensitive properties of gastric vagal afferent fibers in the rat. J Neurophysiol 82 2210-2220.
Prechtl J C, Powley T L 1990 The fiber composition of the abdominal vagus of the rat. Anat Embryol (Berl) 181 101-115.
Schmalbruch H 1986 Fiber composition of the rat sciatic nerve. Anal Rec 215 71-81.
Solomonow M, Eldred E, Lyman J, Foster J 1983 Control of muscle contractile force through indirect high-frequency stimulation. Am J Phys Med 62 71-82.
Tai C, Roppolo J R, de Groat W C 2005c Response of external urethral sphincter to high frequency biphasic electrical stimulation of pudendal nerve. J Urol 174 782-786.
Waataja J, Tweeden K, Honda C 2011 Effects of high-frequency alternating current on axonal conduction through the vagus nerve. J. Neural Eng. 8 (5):056013

1. A system for applying therapy to a target nerve of a subject comprising:
at least two electrodes, each having an impedance of at least 2000 ohms configured to be implanted within a body of the subject and placed at the target nerve,
an implantable component for placement in the body of the subject, the implantable component being configured to generate an electrical signal at a selected voltage or a selected current, wherein the electrical signal is selected to modulate activity on the target nerve, the implantable component being coupled to an implanted antenna;
an external component including an external antenna configured to be placed above the skin layer and adapted to communicate with the implanted antenna communication.

2. The system of claim 1, further comprising an external programmer configured to communicatively couple to the external component, the external programmer being configured to provide therapy instructions to the external component, wherein the external component is configured to send the therapy instructions to the implantable component via the external antenna and the implanted antenna.

3. The system of claim 2, wherein the external programmer includes a personal computer.

4. The system of claim 1, wherein the external component is adapted to be configured into a programming mode when the external programmer is coupled to the external component, wherein the external component does not provide power to the implantable component when configured in the programming mode.

5. The system of claim 1, wherein the electrode has an impedance of 10,000 to 10 megaOhms.

6. The system of claim 5, wherein the electrode is coated with an insulating material that has a resistivity of at least 10² ohm/cm.

7. The system of claim 6, wherein the electrode comprises a coating of acrylic paint, parylene, silicone rubber, polyurethane, polyether ether ketone, polyimide, polyethylene, Teflon, silica/quartz, iridium oxide, tantalum oxide, or aluminum oxide.

8. The system of claim 1, wherein the implantable component comprises circuitry to apply a constant voltage to the electrode.

9. The system of claim 8, wherein the selected voltage is about 20 volts or less.

10. The system of claim 1, wherein the frequency of the electrical signal is selected to downregulate nerve activity.

11. The system of claim 10, wherein the nerve is selected from the group consisting of vagus nerve, cranial nerves, celiac nerve, renal nerve, splanchnic nerve, the celiac plexus, and combinations thereof.

12. The system of claim 10, wherein the electrical signal has a frequency of at least 200 Hz.

13. The system of claim 1, wherein the electrical signal has a pulse width of at least 10 microseconds.

14. The system of claim 1, wherein the external component comprises a user interface that allow for selection of pulse widths.

15. The system of claim 1, wherein the external component comprises a user interface that provides for selection of a voltage.

16. The system of claim 1, wherein the frequency of the electrical signal is selected to upregulate activity on the target nerve.

17. The system of claim 16, wherein the target nerve is glossopharyngeal or baroreceptors.

18. The system of claim 16, wherein the frequency of the electrical signal is less than 200 Hz.

19. A method of treating a disorder in a subject comprising:
Applying at least two electrodes to a target nerve, wherein each electrode has an impedance of at least 2000 ohms and is operatively coupled to an implantable neuroregulator; and
Applying a therapy cycle to the target nerve, wherein the therapy cycle comprises applying an electrical signal at a selected voltage or selected current to the electrode intermittently, and wherein the electrical signal is selected to modulate activity on the target nerve.

20. The method of claim 19, wherein the disorder is selected from the group consisting of obesity, metabolic syndrome, diabetes, hypertension, inflammatory bowel disease, pancreatitis, and bulimia.

21. The method of claim 19, wherein the target nerve is a vagus nerve, a splanchnic nerve, a cranial nerve, celiac nerve, a glossopharyngeal nerve, a celiac nerve or a renal nerve.

22. The method of claim 19, wherein the electrode has an impedance of 10,000 to 10 megaOhms.

23. The method of claim 22, wherein the electrode is coated with an insulating material that has a resistivity of at least 10² ohm/cm.

24. The method of claim 23, wherein the electrode comprises a coating of acrylic paint, paralyene, silicone rubber, polyurethane, polyethylene, polyether ether ketone, polyimide, Teflon, silica/quartz, iridium oxide, tantalum oxide, aluminum oxide, or combinations thereof.

25. The method of claim 19, wherein the electrical signal has a frequency of at least 200 Hz.

26. The method of claim 19, wherein the electrical signal has a pulse width of at least 10 microseconds.

27. The method of claim 19, wherein the frequency of the electrical signal is selected to upregulate activity on the target nerve.

28. The method of claim 27, wherein the target nerve is glossopharyngeal or baroreceptors.

29. The method of claim 27, wherein the frequency of the electrical signal is less than 200 Hz.

30. A system for applying therapy to a target nerve of a subject comprising:
at least two electrodes, each having an impedance of at least 2000 ohms configured to be implanted within a body of the subject and placed at the target nerve, an implantable component for placement in the body of the subject, the implantable component being configured to generate an electrical filed, wherein the electrical field is selected to modulate activity on the target nerve, the implantable component being coupled to an implanted antenna; an external component including an external antenna configured to be placed above the skin layer and adapted to communicate with the implanted antenna across the skin layer through radiofrequency communication.
